(19) United States
(12) Patent Application Publication
(10) Pub. No.: US 2017/0182267 A1
(43) Pub. Date: Jun. 29, 2017

(54) Electronic Vapor Device Spirometer

(71) Applicant: LUNATECH, LLC, Studio City, CA (US)

(72) Inventor: John D. Cameron, Studio City, CA (US)

(21) Appl. No.: 15/391,078

(22) Filed: Dec. 27, 2016

Related U.S. Application Data
(60) Provisional application No. 62/271,835, filed on Dec. 28, 2015.

(51) Int. Cl. A61M 11/04; A61M 15/06; A24F 47/00

(52) U.S. Cl. CPC A61M 11/042; A24F 47/008; A61M 15/06; A61M 2230/40; A61M 2205/502; A61M 2205/18; A61M 2205/583; A61M 2205/3606; A61M 2205/3584

(57) ABSTRACT

Provided are systems, methods, and electronic vapor devices configured to provide spirometer functionality and respiratory medication dispensing which can couple and function symbiotically with a portable electronic communication device.

### CLAIM OF PRIORITY

This application claims priority to provisional patent application Ser. No. 62/271835 filed Dec. 28, 2015, the contents of which are hereby incorporated by reference.

### BACKGROUND

Consumers utilize electronic vapor cigarettes, pipes, and modified vapor devices to enjoy what is commonly known as “vaping.” Vaping is an increasingly popular market segment, which has been, and continues to, steadily gaining market share over the last several years. Various types of personal vaporizers are known in the art. In general, such vaporizers are characterized by heating a solid to a smoldering point, vaporizing a liquid by heat, or nebulizing a liquid by heat and/or by expansion through a nozzle. Such devices are designed to release aromatic materials in the solid or liquid while avoiding high temperatures of combustion and associated formation of tars, carbon monoxide, or other harmful byproducts. Many vaping consumers also suffer from respiratory ailments and utilize a spirometer to determine respiratory function. A spirometer can generally be defined as an instrument for measuring the breathing capacity and/or other bronchial activity of the lungs. Spirometers find wide utilization in the diagnosis of lung and breathing difficulties such as emphysema, asthma and chronic bronchitis. It would be desirable, therefore, to integrate spirometer functionality within electronic vapor devices to improve respiratory function.

### SUMMARY

It is to be understood that both the following general description and the following detailed description are exemplary and explanatory only and are not restrictive. In an aspect, disclosed herein is a system, method and device deployment of an electronic hybrid eSpirometer and respiratory medication dispenser which couples and functions symbiotically to a portable electronic communication device.

In another aspect, provided is an apparatus comprising an air intake and a vapor output, configured for receiving a user exhalation or inhalation. The apparatus can comprise a sensor, coupled to the air intake and the vapor output, configured for determining a spirometry parameter based on the received user exhalation or inhalation. The apparatus can comprise a processor, configured for determining a mixture of vaporizable respiratory medication based on the spirometry parameter. The apparatus can comprise a plurality of containers for storing vaporizable respiratory medication. The apparatus can comprise a mixing element, coupled to the processor, configured for withdrawing a selectable amount of vaporizable respiratory medication from each of the plurality of containers based on the mixture of vaporizable respiratory medication. The apparatus can comprise a mixing chamber coupled to the air intake for receiving air, the mixing element for receiving the selectable amounts of vaporizable respiratory medication. The apparatus can comprise a heating element, coupled to the mixing chamber, configured for heating the selectable amounts of vaporizable respiratory medication and the received air to generate a vapor expelled through the vapor output.

In another aspect, provided is an apparatus comprising a detachable vaporizer/spirometer comprising, an air intake, a vapor output, configured for receiving a user exhalation or inhalation, a sensor, coupled to the air intake and the vapor output, configured for determining a spirometry parameter based on the received user exhalation or inhalation, a processor, configured for determining a vaporizable respiratory medication based on the spirometry parameter, a container for storing the vaporizable respiratory medication, and a mixing chamber coupled to the air intake for receiving air, the container for receiving the vaporizable respiratory medication, and a heating element configured for heating the vaporizable respiratory medication and the received air to generate a heated vapor expelled through the vapor output. The apparatus can comprise an electronic communication device, coupled to the detachable vaporizer/spirometer via an input/output port, comprising, a user input interface for controlling one or more functions of the detachable vaporizer/spirometer.

In another aspect, provided is a method comprising receiving, through a vapor output of an electronic vapor device, a user inhalation or exhalation, generating, by a sensor, first data related to a spirometry parameter based on the user inhalation or exhalation, providing the first data to an electronic communication device, receiving, from the electronic communication device, a command to vaporize a vaporizable respiratory medication, and vaporizing the vaporizable respiratory medication and expelling the resultant vapor through the vapor output.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The features, nature, and advantages of the present disclosure will become more apparent from the detailed description set forth below when taken in conjunction with the drawings, in which like reference characters are used to identify like elements correspondingly throughout the specification and drawings.

FIG. 1 illustrates a block diagram of an exemplary electronic vapor device;

FIG. 2 illustrates an exemplary vaporizer with spirometer functionality;

FIG. 3 illustrates an exemplary vaporizer with spirometer functionality configured for vaporizing a mixture of vaporizable material;

FIG. 4 illustrates an exemplary vaporizer device with spirometer functionality;

FIG. 5 illustrates another exemplary vaporizer with spirometer functionality;

FIG. 6 illustrates another exemplary vaporizer with spirometer functionality;

FIG. 7 illustrates another exemplary vaporizer with spirometer functionality;

FIG. 8 illustrates an exemplary vaporizer with spirometer functionality configured for filtering air;

FIG. 9 illustrates an interface of an exemplary electronic vapor device;

FIG. 10 illustrates another interface of an exemplary electronic vapor device;

FIG. 11 illustrates several interfaces of an exemplary electronic vapor device;

FIG. 12 illustrates an exemplary operating environment;

FIG. 13 illustrates another exemplary operating environment;

FIG. 14 illustrates an exemplary electronic vapor device with spirometer functionality coupled to an electronic communication device; and

FIG. 15 illustrates an exemplary method.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Various aspects are now described with reference to the drawings. In the following description, for purposes of explanation, numerous specific details are set forth in order to provide a thorough understanding of one or more aspects. It may be evident, however, that the various aspects may be practiced without these specific details. In other instances, well-known structures and devices are shown in block diagram form in order to facilitate describing these aspects.

In an aspect, disclosed is a multi (e.g., dual) function electronic vapor (eVapor) device where a user may choose to utilize the eVapor device as either a spirometer to check the users' lung capacity or as a eVapor device capable of delivering asthma and/or lung medications as well as lung wellness formula blended eVapor blend. A device switch enables the user to switch from spirometer mode to eVapor mode.

In an embodiment the eVapor device can be a hybrid component integrated into an electronic communication handset, such as a cellular telephone or electronic tablet device. In another aspect, a spirometer device (e.g., “add on” device) can be coupled with an eVapor device and/or electronic communication device. The spirometer device can be a single rechargeable component, continuous with the electronic communication device or can be removable, portable, and/or disposable/recyclable.

The spirometer device can be “hard wired” to the eVapor device and/or electronic communication device via an electronic connection at the edges of the devices. In an aspect, there can be some designed overlap, depending on which way the devices need to be coupled to optimize continuity and function. The electronic connection between devices can be flush or one or both devices can deploy internal connections into the other device via a protruding port penetrating precise fit positioning of the electronic connection leading from one device inside the other device to engage the electronic connection. A locking system can keep the devices firmly connected, as though the two devices were one.

In an aspect, a mouthpiece which is utilized for eVapor usage may also be utilized to blow into as a spirometer or there may be a separate attachment which is utilized, just for using the spirometer. If a separate attachment is used, then the system software can sense the separate attachment via DLL programming and automatically switch the device to spirometer mode. If the same hardware is utilized for the eVapor device and spirometer device then a switch, dial or other control on the device, a voice command or an interface on the shuttle or communication device can allow the user to select the spirometer (or the eVapor) functionality. The spirometer device, in use, can be triggered by a pressure sensor inside the spirometer device, which gauges the force of the user's breath, once the user breathes into the device. This information obtained by the sensor can be transferred to the processor and correlated via system application software to generate a result, which can be displayed on the spirometer device or eVapor device screen interface.

The system can symbiotically distribute precise amounts of respiratory medication based upon the results (data) of the spirometer device. These data can be shown to the user on a screen that the spirometer test result yielded a certain calibrated dose or no dose, of respiratory medication. The result may also be transmitted to an authorized caregiver (e.g., third party) who can transmit back custom dosage settings of respiratory treatment medication. The spirometer results can be transferred to a software application for dosage recommendation results. The software application can be preloaded with the dosage based upon caregiver specifications correlating to the results or other user, condition and medication data held in the software application with patient specifics input by the caregiver, which may also be auto-implemented via a syncing with one or more health care organizations online treatment and communications portal.

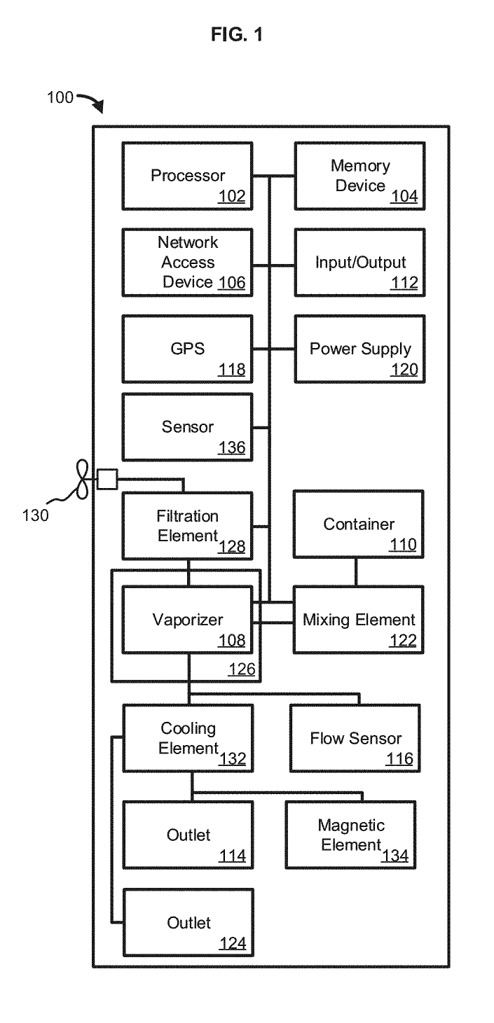

FIG. 1 is a block diagram of an exemplary electronic vapor device 100 as described herein. The electronic vapor device 100 can be, for example, an e-cigarette, an e-cigar, an electronic vapor device, a hybrid electronic communication handset coupled/integrated vapor device, a robotic vapor device, a modified vapor device “mod,” a micro-sized electronic vapor device, a robotic vapor device, and the like. The vapor device 100 can comprise any suitable housing for enclosing and protecting the various components disclosed herein. The vapor device 100 can comprise a processor 102. The processor 102 can be, or can comprise, any suitable microprocessor or microcontroller, for example, a low-power application-specific controller (ASIC) and/or a field programmable gate array (FPGA) designed or programmed specifically for the task of controlling a device as described herein, or a general purpose central processing unit (CPU), for example, one based on 80×86 architecture as designed by Intel™ or AMD™, or a system-on-a-chip as designed by ARM™. The processor 102 can be coupled (e.g., communicatively, operatively, etc . . . ) to auxiliary devices or modules of the vapor device 100 using a bus or other coupling. The vapor device 100 can comprise a power supply 120. The power supply 120 can comprise one or more batteries and/or other power storage device (e.g., capacitor) and/or a port for connecting to an external power supply. For example, an external power supply can supply power to the vapor device 100 and a battery can store at least a portion of the supplied power. The one or more batteries can be rechargeable. The one or more batteries can comprise a lithium-ion battery (including thin film lithium ion batteries), a lithium ion polymer battery, a nickel-cadmium battery, a nickel metal hydride battery, a lead-acid battery, combinations thereof, and the like.

The vapor device 100 can comprise a memory device 104 coupled to the processor 102. The memory device 104 can comprise a random access memory (RAM) configured for storing program instructions and data for execution or processing by the processor 102 during control of the vapor device 100. When the vapor device 100 is powered off or in an inactive state, program instructions and data can be stored in a long-term memory, for example, a non-volatile magnetic optical, or electronic memory storage device (not shown). Either or both of the RAM or the long-term memory can comprise a non-transitory computer-readable medium storing program instructions that, when executed by the processor 102, cause the vapor device 100 to perform all or part of one or more methods and/or operations described herein. Program instructions can be written in any suitable high-level language, for example, C, C++, C# or the Java™, and compiled to produce machine-language code for execution by the processor 102.

In an aspect, the vapor device 100 can comprise a network access device 106 allowing the vapor device 100 to be coupled to one or more ancillary devices (not shown) such as via an access point (not shown) of a wireless telephone network, local area network, or other coupling to a wide area network, for example, the Internet. In that regard, the processor 102 can be configured to share data with the one or more ancillary devices via the network access device 106. The shared data can comprise, for example, usage data and/or operational data of the vapor device 100, a status of the vapor device 100, a status and/or operating condition of one or more the components of the vapor device 100, text to be used in a message, a product order, payment information, and/or any other data. Similarly, the processor 102 can be configured to receive control instructions from the one or more ancillary devices via the network access device 106. For example, a configuration of the vapor device 100, an operation of the vapor device 100, and/or other settings of the vapor device 100, can be controlled by the one or more ancillary devices via the network access device 106. For example, an ancillary device can comprise a server that can provide various services and another ancillary device can comprise a smartphone for controlling operation of the vapor device 100. In some aspects, the smart-phone or another ancillary device can be used as a primary input/output of the vapor device 100 such that data is received by the vapor device 100 from the server, transmitted to the smartphone, and output on a display of the smartphone. In an aspect, data transmitted to the ancillary device can comprise a mixture of vaporizable material and/or instructions to release vapor. For example, the vapor device 100 can be configured to determine a need for the release of vapor into the atmosphere. The vapor device 100 can provide instructions via the network access device 106 to an ancillary device (e.g., another vapor device) to release vapor into the atmosphere.

In an aspect, the vapor device 100 can also comprise an input/output device 112 coupled to one or more of the processor 102, the vaporizer 108, the network access device 106, and/or any other electronic component of the vapor device 100. Input can be received from a user or another device and/or output can be provided to a user or another device via the input/output device 112. The input/output device 112 can comprise any combinations of input and/or output devices such as buttons, knobs, keyboards, touchscreens, displays, light-emitting elements, a speaker, and/or the like. In an aspect, the input/output device 112 can comprise an interface port (not shown) such as a wired interface, for example a serial port, a Universal Serial Bus (USB) port, an Ethernet port, or other suitable wired connection. The input/output device 112 can comprise a wireless interface (not shown), for example a transceiver using any suitable wireless protocol, for example WiFi (IEEE 802.11), Bluetooth®, infrared, or other wireless standard. For example, the input/output device 112 can communicate with a smartphone via Bluetooth® such that the inputs and outputs of the smartphone can be used by the user to interface with the vapor device 100. In an aspect, the input/output device 112 can comprise a user interface. The user interface user interface can comprise at least one of lighted signal lights, gauges, boxes, forms, check marks, avatars, visual images, graphic designs, lists, active calibrations or calculations, 2D interactive fractal designs, 3D fractal designs, 2D and/or 3D representations of vapor devices and other interface system functions.

In an aspect, the input/output device 112 can comprise a touchscreen interface and/or a biometric interface. For example, the input/output device 112 can include controls that allow the user to interact with and input information and commands to the vapor device 100. For example, with respect to the embodiments described herein, the input/output device 112 can comprise a touch screen display. The input/output device 112 can be configured to provide the content of the exemplary screen shots shown herein, which are presented to the user via the functionality of a display. User inputs to the touch screen display are processed by, for example, the input/output device 112 and/or the processor 102. The input/output device 112 can also be configured to process new content and communications to the system 100. The touch screen display can provide controls and menu selections, and process commands and requests. Application and content objects can be provided by the touch screen display. The input/output device 112 and/or the processor 102 can receive and interpret commands and other inputs, interface with the other components of the vapor device 100 as required. In an aspect, the touch screen display can enable a user to lock, unlock, or partially unlock or lock, the vapor device 100. The vapor device 100 can be transitioned from an idle and locked state into an open state by, for example, moving or dragging an icon on the screen of the vapor device 100, entering in a password/passcode, and the like. The input/output device 112 can thus display information to a user such as a puff count, an amount of vaporizable material remaining in the container 110, battery remaining, signal strength, combinations thereof, and the like.

In an aspect, the input/output device 112 can comprise an audio user interface. A microphone can be configured to receive audio signals and relay the audio signals to the input/output device 112. The audio user interface can be any interface that is responsive to voice or other audio commands. The audio user interface can be configured to cause an action, activate a function, etc, by the vapor device 100 (or another device) based on a received voice (or other audio) command. The audio user interface can be deployed directly on the vapor device 100 and/or via other electronic devices (e.g., electronic communication devices such as a smartphone, a smart watch, a tablet, a laptop, a dedicated audio user interface device, and the like). The audio user interface can be used to control the functionality of the vapor device 100. Such functionality can comprise, but is not limited to, custom mixing of vaporizable material (e.g., eLiquids) and/or ordering custom made eLiquid combinations via an eCommerce service (e.g., specifications of a user's custom flavor mix can be transmitted to an eCommerce service, so that an eLiquid provider can mix a custom eLiquid cartridge for the user). The user can then reorder the custom flavor mix anytime or even send it to friends as a present, all via the audio user interface. The user can also send via voice command a mixing recipe to other users. The other users can utilize the mixing recipe (e.g., via an electronic vapor device having multiple chambers for eLiquid) to sample the same mix via an auto-order to the other users' devices to create the received mixing recipe. A custom mix can be given a title by a user and/or can be defined by parts (e.g., one part liquid. A and two parts liquid B). The audio user interface can also be utilized to create and send a custom message to other users, to join eVapor clubs, to receive eVapor chart information, and to conduct a wide range of social networking, location services and eCommerce activities. The audio user interface can be secured via a password (e.g., audio password) which features at least one of tone recognition, other voice quality recognition and, in one aspect, can utilize at least one special cadence as part of the audio password.

The input/output device 112 can be configured to interface with other devices, for example, exercise equipment, computing equipment, communications devices and/or other vapor devices, for example, via a physical or wireless connection. The input/output device 112 can thus exchange data with the other equipment. A user may sync their vapor device 100 to other devices, via programming attributes such as mutual dynamic link library (DLL) ‘hooks’. This enables a smooth exchange of data between devices, as can a web interface between devices. The input/output device 112 can be used to upload one or more profiles to the other devices. Using exercise equipment as an example, the one or more profiles can comprise data such as workout routine data (e,g., timing, distance, settings, heart rate, etc . . . ) and vaping data (e.g., eLiquid mixture recipes, supplements, vaping timing, etc . . . ). Data from usage of previous exercise sessions can be archived and shared with new electronic vapor devices and/or new exercise equipment so that history and preferences may remain continuous and provide for simplified device settings, default settings, and recommended settings based upon the synthesis of current and archival data.

In an aspect, the vapor device 100 can comprise a vaporizer 108. The vaporizer 108 can be coupled to one or more containers 110. Each of the one or more containers 110 can be configured to hold one or more vaporizable or non-vaporizable materials. The vaporizer 108 can receive the one or more vaporizable or non-vaporizable materials from the one or more containers 110 and heat the one or more vaporizable or non-vaporizable materials until the one or more vaporizable or non-vaporizable materials achieve a vapor state. In various embodiments, instead of heating the one or more vaporizable or non-vaporizable materials, the vaporizer 108 can nebulize or otherwise cause the one or more vaporizable or non-vaporizable materials in the one or more containers 110 to reduce in size into particulates. In various embodiments, the one or more containers 110 can comprise a compressed liquid that can be released to the vaporizer 108 via a valve or another mechanism. In various embodiments, the one or more containers 110 can comprise a wick (not shown) through which the one or more vaporizable or non-vaporizable materials is drawn to the vaporizer 108. The one or more containers 110 can be made of any suitable structural material, such as, an organic polymer, metal, ceramic, composite, or glass material.

In an aspect, the vapor device 100 can comprise a mixing element 122. The mixing element 122 can be coupled to the processor 102 to receive one or more control signals. The one or more control signals can instruct the mixing element 122 to withdraw specific amounts of fluid from the one or more containers 110. The mixing element can, in response to a control signal from the processor 102, withdraw select quantities of vaporizable material in order to create a customized mixture of different types of vaporizable material. The liquid withdrawn by the mixing element 122 can be provided to the vaporizer 108. In an aspect, the vaporizable material can comprise one or more respiratory medications.

In an aspect, input from the input/output device 112 can be used by the processor 102 to cause the vaporizer 108 to vaporize the one or more vaporizable or non-vaporizable materials. For example, a user can press a button, causing the vaporizer 108 to start vaporizing the one or more vaporizable or non-vaporizable materials. A user can then draw on an outlet 114 to inhale the vapor. In various aspects, the processor 102 can control vapor production and flow to the outlet 114 based on data detected by a flow sensor 116. For example, as a user draws on the outlet 114, the flow sensor 116 can detect the resultant pressure and provide a signal to the processor 102. In response, the processor 102 can cause the vaporizer 108 to begin vaporizing the one or more vaporizable or non-vaporizable materials, terminate vaporizing the one or more vaporizable or non-vaporizable materials, and/or otherwise adjust a rate of vaporization of the one or more vaporizable or non-vaporizable materials. In another aspect, the vapor can exit the vapor device 100 through an outlet 124. The outlet 124 differs from the outlet 114 in that the outlet 124 can be configured to distribute the vapor into the local atmosphere, rather than being inhaled by a user. In an aspect, vapor exiting the outlet 124 can be at least one of aromatic, medicinal, recreational, and/or wellness related.

In another aspect, the vapor device 100 can comprise a piezoelectric dispersing element. In some aspects, the piezoelectric dispersing element can be charged by a battery, and can be driven by a processor on a circuit board. The circuit board can be produced using a polyimide such as Kapton, or other suitable material. The piezoelectric dispersing element can comprise a thin metal disc which causes dispersion of the fluid fed into the dispersing element via the wick or other soaked piece of organic material through vibration. Once in contact with the piezoelectric dispersing element, the vaporizable material (e.g., fluid) can be vaporized (e.g., turned into vapor or mist) and the vapor can be dispersed via a system pump and/or a sucking action of the user. In some aspects, the piezoelectric dispersing element can cause dispersion of the vaporizable material by producing ultrasonic vibrations. An electric field applied to a piezoelectric material within the piezoelectric element can cause ultrasonic expansion and contraction of the piezoelectric material, resulting in ultrasonic vibrations to the disc. The ultrasonic vibrations can cause the vaporizable material to disperse, thus forming a vapor or mist from the vaporizable material.

In some aspects, the connection between a power supply and the piezoelectric dispersing element can be facilitated using one or more conductive coils. The conductive coils can provide an ultrasonic power input to the piezoelectric dispersing element. For example, the signal carried by the coil can have a frequency of approximately 107.8 kHz. In some aspects, the piezoelectric dispersing element can comprise a piezoelectric dispersing element that can receive the ultrasonic signal transmitted from the power supply through the coils, and can cause vaporization of the vaporizable liquid by producing ultrasonic vibrations. An ultrasonic electric field applied to a piezoelectric material within the piezoelectric element causes ultrasonic expansion and contraction of the piezoelectric material, resulting in ultrasonic vibrations according to the frequency of the signal. The vaporizable liquid can be vibrated by the ultrasonic energy produced by the piezoelectric dispersing element, thus causing dispersal and/or atomization of the liquid. In an aspect, the vapor device 100 can be configured to permit a user to select between using a heating element of the vaporizer 108 or the piezoelectric dispersing element. In another aspect, the vapor device 100 can be configured to permit a user to utilize both a heating element of the vaporizer 108 and the piezoelectric dispersing element.

In an aspect, the vapor device 100 can comprise a heating casing 126. The heating casing 126 can enclose one or more of the container 110, the vaporizer 108, and/or the outlet 114. In a further aspect, the heating casing 126 can enclose one or more components that make up the container 110, the vaporizer 108, and/or the outlet 114. The heating casing 126 can be made of ceramic, metal, and/or porcelain. The heating casing 126 can have varying thickness. In an aspect, the heating casing 126 can be coupled to the power supply 120 to receive power to heat the heating casing 126. In another aspect, the heating casing 126 can be coupled to the vaporizer 108 to heat the heating casing 126. In another aspect, the heating casing 126 can serve an insulation role.

In an aspect, the vapor device 100 can comprise a filtration element 128. The filtration element 128 can be configured to remove (e.g., filter, purify, etc) contaminants from air entering the vapor device 100. The filtration element 128 can optionally comprise a fan 130 to assist in delivering air to the filtration element 128. The vapor device 100 can be configured to intake air into the filtration element 128, filter the air, and pass the filtered air to the vaporizer 108 for use in vaporizing the one or more vaporizable or non-vaporizable materials. In another aspect, the vapor device 100 can be configured to intake air into the filtration element 128, filter the air, and bypass the vaporizer 108 by passing the filtered air directly to the outlet 114 for inhalation by a user.

In an aspect, the filtration element 128 can comprise cotton, polymer, wool, satin, meta materials and the like. The filtration element 128 can comprise a filter material that at least one airborne particle and/or undesired gas by a mechanical mechanism, an electrical mechanism, and/or a chemical mechanism. The filter material can comprise one or more pieces of a filter fabric that can filter out one or more airborne particles and/or gasses. The filter fabric can be a woven and/or non-woven material. The filter fabric can be made from natural fibers (e.g., cotton, wool, etc.) and/or from synthetic fibers (e.g., polyester, nylon, polypropylene, etc.). The thickness of the filter fabric can be varied depending on the desired filter efficiencies and/or the region of the apparel where the filter fabric is to be used. The filter fabric can be designed to filter airborne particles and/or gasses by mechanical mechanisms (e.g., weave density), by electrical mechanisms (e.g., charged fibers, charged metals, etc.), and/or by chemical mechanisms (e.g., absorptive charcoal particles, adsorptive materials, etc.). In as aspect, the filter material can comprise electrically charged fibers such as, but not limited to, FILTRETE by 3M. In another aspect, the filter material can comprise a high density material similar to material used for medical masks which are used by medical personnel in doctors' offices, hospitals, and the like. In an aspect, the filter material can be treated with an anti-bacterial solution and/or otherwise made from anti-bacterial materials. In another aspect, the filtration element 128 can comprise electrostatic plates, ultraviolet light, a HEPA filter, combinations thereof, and the like.

In an aspect, the vapor device 100 can comprise a cooling element 132. The cooling element 132 can be configured to cool vapor exiting the vaporizer 108 prior to passing through the outlet 114. The cooling element 132 can cool vapor by utilizing air or space within the vapor device 100. The air used by the cooling element 132 can be either static (existing in the vapor device 100) or drawn into an intake and through the cooling element 132 and the vapor device 100. The intake can comprise various pumping, pressure, fan, or other intake systems for drawing air into the cooling element 132. In an aspect, the cooling element 132 can reside separately or can be integrated the vaporizer 108. The cooling element 132 can be a single cooled electronic element within a tube or space and/or the cooling element 132 can be configured as a series of coils or as a grid like structure. The materials for the cooling element 132 can be metal, liquid, polymer, natural substance, synthetic substance, air, or any combination thereof. The cooling element 132 can be powered by the power supply 120, by a separate battery (not shown), or other power source (not shown) including the use of excess heat energy created by the vaporizer 108 being converted to energy used for cooling by virtue of a small turbine or pressure system to convert the energy. Heat differentials between the vaporizer 108 and the cooling element 132 can also be converted to energy utilizing commonly known geothermal energy principles.

In an aspect, the vapor device 100 can comprise a magnetic element 134. For example, the magnetic element 134 can comprise an electromagnet, a ceramic magnet, a ferrite magnet, and/or the like. The magnetic element 134 can be configured to apply a magnetic field to air as it is brought into the vapor device 100, in the vaporizer 108, and/or as vapor exits the outlet 114.

The input/output device 112 can be used to select whether vapor exiting the outlet 114 should be cooled or not cooled and/or heated or not heated and/or magnetized or not magnetized. For example, a user can use the input/output device 112 to selectively cool vapor at times and not cool vapor at other times. The user can use the input/output device 112 to selectively heat vapor at times and not heat vapor at other times. The user can use the input/output device 112 to selectively magnetize vapor at times and not magnetize vapor at other times. The user can further use the input/output device 112 to select a desired smoothness, temperature, and/or range of temperatures. The user can adjust the temperature of the vapor by selecting or clicking on a clickable setting on a part of the vapor device 100. The user can use, for example, a graphical user interface (GUI) or a mechanical input enabled by virtue of clicking a rotational mechanism at either end of the vapor device 100.

In an aspect, cooling control can be set within the vapor device 100 settings via the processor 102 and system software (e.g., dynamic linked libraries). The memory 104 can store settings. Suggestions and remote settings can be communicated to and/or from the vapor device 1.00 via the input/output device 112 and/or the network access device 106. Cooling of the vapor can be set and calibrated between heating and cooling mechanisms to what is deemed an ideal temperature by the manufacturer of the vapor device 100 for the vaporizable material. For example, a temperature can be set such that resultant vapor delivers the coolest feeling to the average user but does not present any health risk to the user by virtue of the vapor being too cold, including the potential for rapid expansion of cooled vapor within the lungs and the damaging of tissue by vapor which has been cooled to a temperature which may cause frostbite like symptoms.

In an aspect, the vapor device 100 can be configured to receive air, smoke, vapor or other material and analyze the contents of the air, smoke, vapor or other material using one or more sensors 136 in order to at least one of analyze, classify, compare, validate, refute, and/or catalogue the same. A result of the analysis can be, for example, an identification of at least one of medical, recreational, homeopathic, olfactory elements, spices, other cooking ingredients, ingredients analysis from food products, fuel analysis, pharmaceutical analysis, genetic modification testing analysis, dating, fossil and/or relic analysis and the like. The vapor device 100 can pass utilize, for example, mass spectrometry, PH testing, genetic testing, particle and/or cellular testing, sensor based testing and other diagnostic and wellness testing either via locally available components or by transmitting data to a remote system for analysis.

In an aspect, a user can create a custom scent by using the vapor device 100 to intake air elements, where the vapor device 100 (or third-party networked device) analyzes the olfactory elements and/or biological elements within the sample and then formulates a replica scent within the vapor device 100 (or third-party networked device) that can be accessed by the user instantly, at a later date, with the ability to purchase this custom scent from a networked ecommerce portal.

In another aspect, the one or more sensors 136 can be configured to sense negative environmental conditions (e.g., adverse weather, smoke, fire, chemicals (e.g., such as CO2 or formaldehyde), adverse pollution, and/or disease outbreaks, and the like). The one or more sensors 136 can comprise one or more of, a biochemical/chemical sensor, a thermal sensor, a radiation sensor, a mechanical sensor, an optical sensor, a mechanical sensor, a magnetic sensor, an electrical sensor, combinations thereof and the like. The biochemical/chemical sensor can be configured to detect one or more biochemical/chemicals causing a negative environmental condition such as, but not limited to, smoke, a vapor, a gas, a liquid, a solid, an odor, combinations thereof, and/or the like. The biochemical/chemical sensor can comprise one or more of a mass spectrometer, a conducting/nonconducting regions sensor, a SAW sensor, a quartz microbalance sensor, a conductive composite sensor, a chemiresitor, a metal oxide gas sensor, an organic gas sensor, a MOSFET, a piezoelectric device, an infrared sensor, a sintered metal oxide sensor, a Pd-gate MOSFET, a metal FET structure, a electrochemical cell, a conducting polymer sensor, a catalytic gas sensor, an organic semiconducting gas sensor, a solid electrolyte gas sensors, a piezoelectric quartz crystal sensor, and/or combinations thereof.

The thermal sensor can be configured to detect temperature, heat, heat flow, entropy, heat capacity, combinations thereof, and the like. Exemplary thermal sensors include, but are not limited to, thermocouples, such as a semiconducting thermocouples, noise thermometry, thermoswitches, thermistors, metal thermoresistors, semiconducting thermoresistors, thermodiodes, thermotransistors, calorimeters, thermometers, indicators, and fiber optics.

The radiation sensor can be configured to detect gamma rays, X-rays, ultra-violet rays, visible, infrared, microwaves and radio waves. Exemplary radiation sensors are suitable for use in the present invention that include, but are not limited to, nuclear radiation microsensors, such as scintillation counters and solid state detectors, ultra-violet, visible and near infrared radiation microsensors, such as photoconductive cells, photodiodes, phototransistors, infrared radiation microsensors, such as photoconductive IR sensors and pyroelectric sensors.

The optical sensor can be configured to detect visible, near infrared, and infrared waves. The mechanical sensor can be configured to detect displacement, velocity, acceleration, force, torque, pressure, mass, flow, acoustic wavelength, and amplitude. Exemplary mechanical sensors are suitable for use in the present invention and include, but are not limited to, displacement microsensors, capacitive and inductive displacement sensors, optical displacement sensors, ultrasonic displacement sensors, pyroelectric, velocity and flow microsensors, transistor flow microsensors, acceleration microsensors, piezoresistive microaccelerometers, force, pressure and strain microsensors, and piezoelectric crystal sensors. The magnetic sensor can be configured to detect magnetic field, flux, magnetic moment, magnetization, and magnetic permeability. The electrical sensor can be configured to detect charge, current, voltage, resistance, conductance, capacitance, inductance, dielectric permittivity, polarization and frequency.

Upon sensing a negative environmental condition, the one or more sensors 136 can provide data to the processor 102 to determine the nature of the negative environmental condition and to generate/transmit one or more alerts based on the negative environmental condition. The one or more alerts can be deployed to the vapor device 100 user's wireless device and/or synced accounts. For example, the network device access device 106 can be used to transmit the one or more alerts directly (e.g., via Bluetooth®) to a user's smartphone to provide information to the user. In another aspect, the network access device 106 can be used to transmit sensed information and/or the one or more alerts to a remote server for use in syncing one or more other devices used by the user (e.g., other vapor devices, other electronic devices (smartphones, tablets, laptops, etc . . . ). In another aspect, the one or more alerts can be provided to the user of the vapor device 100 via vibrations, audio, colors, and the like deployed from the mask, for example through the input/output device 112. For example, the input/output device 112 can comprise a small vibrating motor to alert the user to one or more sensed conditions via tactile sensation. In another example, the input/output device 112 can comprise one or more LED's of various colors to provide visual information to the user. In another example, the input/output device 112 can comprise one or more speakers that can provide audio information to the user. For example, various patterns of beeps, sounds, and/or voice recordings can be utilized to provide the audio information to the user. In another example, the input/output device 112 can comprise an LCD screen/touchscreen that provides a summary and/or detailed information regarding the negative environmental condition and/or the one or more alerts.

In another aspect, upon sensing a negative environmental condition, the one or more sensors 136 can provide data to the processor 102 to determine the nature of the negative environmental condition and to provide a recommendation for mitigating and/or to actively mitigate the negative environmental condition. Mitigating the negative environmental conditions can comprise, for example, applying a filtration system, a fan, a fire suppression system, engaging a HVAC system, and/or one or more vaporizable and/or non-vaporizable materials. The processor 102 can access a database stored in the memory device 104 to make such a determination or the network device 106 can be used to request information from a server to verify the sensor findings. In an aspect, the server can provide an analysis service to the vapor device 100. For example, the server can analyze data sent by the vapor device 100 based on a reading from the one or more sensors 136. The server can determine and transmit one or more recommendations to the vapor device 100 to mitigate the sensed negative environmental condition. The vapor device 100 can use the one or more recommendations to activate a filtration system, a fan, a fire suppression system engaging a HVAC system, and/or to vaporize one or more vaporizable or non-vaporizable materials to assist in countering effects from the negative environmental condition.

In an aspect, the one or more sensors 136 can comprise a pressure sensor configured for measuring a volume and/or rate of air inspired and/or expired by a user's lungs. In an aspect, the flow sensor 116 can be configured to permit the vapor device 100 to function as a spirometer. The one or more sensors 136 can be configured to measure one or more spirometry parameters. For example, a parameter referred to as peak respiratory flow or peak expiratory flow (PEF). Peak expiratory flow (“peak flow”) can be defined as the maximum flow rate recorded during a forced expiration of air from the lungs. Other spirometry parameters can be measured such as, Tidal Volume (TV) (the amount of air inhaled or exhaled during a single breath without forced conditions); Inspiratory Reserve Volume (IRV) (the maximum additional air that can be inhaled at the end of a normal inspiration); Expiratory Reserve Volume (ERV) (refers to the maximum volume of air that can be exhaled at the end of a normal expiration); Vital Capacity (VC) (the maximum amount of air that can be expelled from a person's lungs after a maximum inspiration, the vital capacity is equal to the sum of IRV, ERV, and TV); Forced Vital Capacity (FVC) (the volume of air that can be blown out by a person at a maximal speed and effort after a full inspiration); Forced Expiratory Volume in 1 second (FEV1) (represents the maximum volume of air that can be exhaled in a forced way in the first second, after taking a deep breath); Forced Inspiratory Vital Capacity (FIVC) (the maximum air volume that can be inhaled); Peak Inspiratory Flow (PIF) (the forced maximum flow that can be achieved during inhalation); and/or Peak Expiratory Flow (PEF) (the maximum air flow that can be forced during exhalation).

In an aspect, a method to measure flow can be based on laws of fluid dynamics, for example, the Venturi effect according to which when a fluid passes from a wider to a narrower section of a pipe, the pressure of the fluid reduces while the velocity increases. The velocity and pressure of the fluid change to satisfy mass conservation is regulated by the “Venturi effect equation”:

$${P}_{1} - {P}_{2} = \frac{d}{2} ( {v}_{2}^{2} - {v}_{1}^{2} )$$

where P₁ and P₂ are pressures, p is the density of the fluid and v₁-v₂ are the velocities before and after entering the pipe constriction respectively. Considering Q=v₁A₁=v₂A₂ where Q is flow rate, v is velocity and A is area, there are some operations that can be performed:

$$Q = {v}_{1} {A}_{1} ; {v}_{1} = \sqrt{{v}_{2}^{2} - \frac{2 ( {P}_{1} - {P}_{2} )}{d}} ;$$

$${v}_{1} = \sqrt{{v}_{2}^{2} - \frac{2 ( {P}_{1} - {P}_{2} )}{d}} ;$$

$$Q = {A}_{1} \sqrt{{v}_{2}^{2} - \frac{2 Δ P}{d}}$$

Thus it is possible to find the flow rate in the sensor if the differential pressure between the sections of the different diameters and the response of the tube to the changes in velocity is known. The outlet 114 and an air intake of the vapor device 100 can comprise a tube with two air ways to transmit the air pressure to the pressure sensor that handles the conversion of the signal. The user breathes the outlet 114, which can comprise a flow restriction mechanism that enables the air to flow just through one of the air ways in the tube, depending on whether the user is inhaling or exhaling.

A user's respiratory condition can be monitored by measuring peak flow within the vapor device 100. The user can exhale into the vapor device 100 through the outlet 114 and/or the outlet 124. The one or more sensors 136 can be coupled to the outlet 114 and/or the outlet 124 to measure the volume and/or rate of the exhalation. In another aspect the user can inhale from the vapor device through the outlet 114 and/or the outlet 124. The one or more sensors 136 can be coupled to the outlet 114 and/or the outlet 124 to measure the volume and/or rate of the inspiration. In an aspect, the one or more sensors 136 can be configured to calculate the air flow from a pressure difference measured across an obstruction in a flow channel extending from the outlet 114 and/or the outlet 124 and an air intake. A differential pressure sensor can be connected to two outlets (e.g., the air intake and the outlet 114 and/or the air intake and the outlet 124) on the flow channel on either side of the flow obstruction. The obstruction may be a restriction in the flow channel or a fine wire mesh or ceramic screen. During a user expiration and/or inspiration, the electrical signals from the pressure sensor can be amplified, converted from analog to digital by an A/D converter, and stored in the memory device 104 by the processor 102.

In order to determine if a peak flow result is reliable, the vapor device 100 can be configured to observe the overall flow versus time during the entire breath. The vapor device 100 can store flow time data along with a time/date stamp. The vapor device 100 can perform an analysis for each expiration/inspiration and can display these results on a display via the input/output 112 of the vapor device 100.

In another aspect, the vapor device 100 can couple with an electronic communication device via the input/output 112. The vapor device 100 can thus interface with the electronic communication device, permitting the user to transfer data from the vapor device 100 and display the results on a larger external screen with additional software/processing capabilities. Since the data from the entire flow can be captured by the vapor device 100, additional analysis may also be performed on the data, either within the vapor device 100 itself or by the electronic communication device. En another aspect, the captured flow data can be transmitted to a remote computing device via the network access device 106 for further analysis. For example, the remote computing device can recommend one or more mixtures of fluid be vaporized to treat a condition indicated by the flow data.

In another aspect, the flow data can be analyzed by the processor 102, analyzed by the electronic communication device coupled to the vapor device 100, and/or analyzed by a remote computing device. Analysis of the flow data can determine one or more formulations of respiratory medication that should be delivered to the user based on the flow data. The one or more formulations can be created by the mixing element 122 by drawing in the specified quantities of respiratory medication from the one or more containers 110. The resulting respiratory medication can then be vaporized and delivered to the user via the outlet 114 and/or the outlet 124 as part of normal vapor operation. In aspect, the vapor device 100 can operate in a spirometer mode and a vaping mode. In the spirometer mode, the vapor device 100 can perform spirometer measurements and present the result to the user. In the vaping mode, the vapor device 100 can perform normal vaping functions, including the delivery of vaporized respiratory medication. The vapor device 100 can switch between the spirometer mode and the vaping mode via the input/output 112. In another aspect, the vapor device 100 can switch between the spirometer mode and the vaping mode via the input/output 112 by receiving a switch command from a coupled electronic communication device.

In another aspect, one or more functions of the vapor device 100 can be activated/de-activated to ensure optimal delivery of the respiratory medication. For example, the cooling element 132, the heating casing 126, and/or the magnetic element 134 can be activated/de-activated to ensure optimal delivery of the vaporized respiratory medication.

In an aspect, the vapor device 100 can comprise a global positioning system (GPS) unit 118. The GPS 118 can detect a current location of the device 100. In some aspects, a user can request access to one or more services that rely on a current location of the user. For example, the processor 102 can receive location data from the GPS 118, convert it to usable data, and transmit the usable data to the one or more services via the network access device 106. GPS unit 118 can receive position information from a constellation of satellites operated by the U.S. Department of Defense. Alternately, the GPS unit 118 can be a GLONASS receiver operated by the Russian. Federation Ministry of Defense, or any other positioning device capable of providing accurate location information (for example, LORAN, inertial navigation, and the like). The GPS unit 118 can contain additional logic, either software, hardware or both to receive the Wide Area Augmentation System (WARS) signals, operated by the Federal Aviation Administration, to correct dithering errors and provide the most accurate location possible. Overall accuracy of the positioning equipment subsystem containing WAAS is generally in the two meter range.

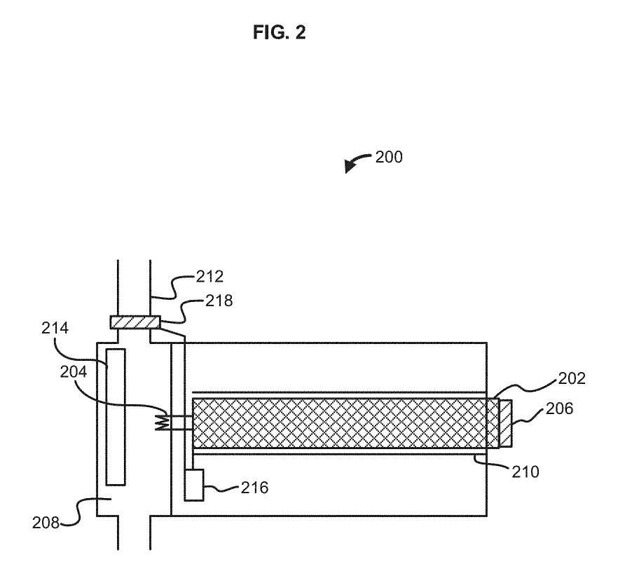

FIG. 2 illustrates an exemplary vaporizer 200. The vaporizer 200 can be, for example, an e-cigarette, an e-cigar, an electronic vapor device, a hybrid electronic communication handset coupled/integrated vapor device, a robotic vapor device, a modified vapor device “mod,” a micro-sized electronic vapor device, a robotic vapor device, and the like. The vaporizer 200 can be used internally of the vapor device 100 or can be a separate device. For example, the vaporizer 200 can be used in place of the vaporizer 108.

The vaporizer 200 can comprise or be coupled to one or more containers 202 containing a vaporizable material, for example a fluid. For example, coupling between the vaporizer 200 and the one or more containers 202 can be via a wick 204, via a valve, or by some other structure. Coupling can operate independently of gravity, such as by capillary action or pressure drop through a valve. The vaporizer 200 can be configured to vaporize the vaporizable material from the one or more containers 202 at controlled rates in response to mechanical input from a component of the vapor device 100, and/or in response to control signals from the processor 102 or another component. Vaporizable material (e.g., fluid) can be supplied by one or more replaceable cartridges 206. In an aspect the vaporizable material can comprise aromatic elements. In an aspect, the aromatic elements can be medicinal, recreational, and/or wellness related. The aromatic element can include, but is not limited to, at least one of lavender or other floral aromatic eLiquids, mint, menthol, herbal soil or geologic, plant based, name brand perfumes, custom mixed perfume formulated inside the vapor device 100 and aromas constructed to replicate the smell of different geographic places, conditions, and/or occurrences. For example, the smell of places may include specific or general sports venues, well known travel destinations, the mix of one's own personal space or home. The smell of conditions may include, for example, the smell of a pet, a baby, a season, a general environment (e.g., a forest), a new car, a sexual nature (e.g., musk, pheromones, etc . . . ). The one or more replaceable cartridges 206 can contain the vaporizable material. If the vaporizable material is liquid, the cartridge can comprise the wick 204 to aid in transporting the liquid to a mixing chamber 208. In the alternative, some other transport mode can be used. Each of the one or more replaceable cartridges 206 can be configured to fit inside and engage removably with a receptacle (such as the container 202 and/or a secondary container) of the vapor device 100. In an alternative, or in addition, one or more fluid containers 210 can be fixed in the vapor device 100 and configured to be refillable. In an aspect, one or more materials can be vaporized at a single time by the vaporizer 200. For example, some material can be vaporized and drawn through an exhaust port 212 and/or some material can be vaporized and exhausted via a smoke simulator outlet (not shown).

In operation, a heating element 214 can vaporize or nebulize the vaporizable material in the mixing chamber 208, producing an inhalable vapor/mist that can be expelled via the exhaust port 212. In an aspect, the heating element 214 can comprise a heater coupled to the wick (or a heated wick) 204 operatively coupled to (for example, in fluid communication with) the mixing chamber 210. The heating element 214 can comprise a nickel-chromium wire or the like, with a temperature sensor (not shown) such as a thermistor or thermocouple. Within definable limits, by controlling power to the wick 204, a rate of vaporization can be independently controlled. A multiplexer 216 can receive power from any suitable source and exchange data signals with a processor, for example, the processor 102 of the vapor device 100, for control of the vaporizer 200. At a minimum, control can be provided between no power (off state) and one or more powered states. Other control mechanisms can also be suitable.

In another aspect, the vaporizer 200 can comprise a piezoelectric dispersing element. In some aspects, the piezoelectric dispersing element can be charged by a battery, and can be driven by a processor on a circuit board. The circuit board can be produced using a polyimide such as Kapton, or other suitable material. The piezoelectric dispersing element can comprise a thin metal disc which causes dispersion of the fluid fed into the dispersing element via the wick or other soaked piece of organic material through vibration. Once in contact with the piezoelectric dispersing element, the vaporizable material (e.g., fluid) can be vaporized (e.g., turned into vapor or mist) and the vapor can be dispersed via a system pump and/or a sucking action of the user. In some aspects, the piezoelectric dispersing element can cause dispersion of the vaporizable material by producing ultrasonic vibrations. An electric field applied to a piezoelectric material within the piezoelectric element can cause ultrasonic expansion and contraction of the piezoelectric material, resulting in ultrasonic vibrations to the disc. The ultrasonic vibrations can cause the vaporizable material to disperse, thus forming a vapor or mist from the vaporizable material.

In an aspect, the vaporizer 200 can be configured to permit a user to select between using the heating element 214 or the piezoelectric dispersing element. In another aspect, the vaporizer 200 can be configured to permit a user to utilize both the heating element 214 and the piezoelectric dispersing element,

In some aspects, the connection between a power supply and the piezoelectric dispersing element can be facilitated using one or more conductive coils. The conductive coils can provide an ultrasonic power input to the piezoelectric dispersing element. For example, the signal carried by the coil can have a frequency of approximately 107.8 kHz. In some aspects, the piezoelectric dispersing element can comprise a piezoelectric dispersing element that can receive the ultrasonic signal transmitted from the power supply through the coils, and can cause vaporization of the vaporizable liquid by producing ultrasonic vibrations. An ultrasonic electric field applied to a piezoelectric material within the piezoelectric element causes ultrasonic expansion and contraction of the piezoelectric material, resulting in ultrasonic vibrations according to the frequency of the signal. The vaporizable liquid can be vibrated by the ultrasonic energy produced by the piezoelectric dispersing element, thus causing dispersal and/or atomization of the liquid.

In an aspect, the vaporizer 200 can comprise a pressure sensor 218 configured for measuring a volume and/or rate of air inspired and/or expired by a user's lungs. In an aspect, the pressure sensor 218 can be configured to permit the vaporizer 200 to function as a spirometer. The user can exhale into the vaporizer 200 through the outlet 212. The pressure sensor 218 can be coupled to the outlet 212 and measure the volume and/or rate of the exhalation. In another aspect, the user can inhale from the vaporizer 200 through the outlet 212. The pressure sensor 218 can be coupled to the outlet 212 to measure the volume and/or rate of the inspiration. In an aspect, the pressure sensor 218 can be configured to calculate the air flow from a pressure difference measured across an obstruction in a flow channel extending from the outlet 212 an air intake. During a user expiration and/or inspiration, electrical signals from the pressure sensor 218 can be amplified, converted from analog to digital by an A/D converter, and stored in a memory device.

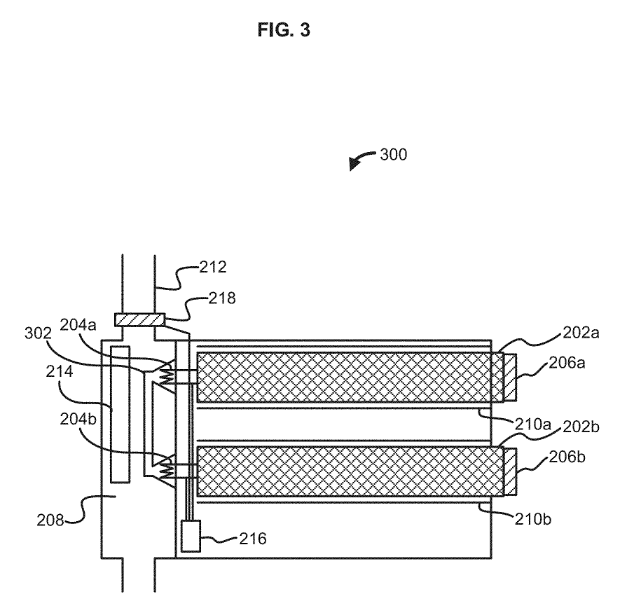

FIG. 3 illustrates a vaporizer 300 that comprises the elements of the vaporizer 200 with two containers 202a and 202b containing a vaporizable material, for example a fluid. In an aspect, the fluid can be the same fluid in both containers or the fluid can be different in each container. In an aspect the fluid can comprise aromatic elements. The aromatic element can include, but is not limited to, at least one of lavender or other floral aromatic eLiquids, mint, menthol, herbal soil or geologic, plant based, name brand perfumes, custom mixed perfume formulated inside the vapor device 100 and aromas constructed to replicate the smell of different geographic places, conditions, and/or occurrences. For example, the smell of places may include specific or general sports venues, well known travel destinations, the mix of one's own personal space or home. The smell of conditions may include, for example, the smell of a pet, a baby, a season, a general environment (e.g., a forest), a new car, a sexual nature (e.g., musk, pheromones, etc . . . ). Coupling between the vaporizer 200 and the container 202a and the container 202b can be via a wick 204a and a wick 204b, respectively, via a valve, or by some other structure. Coupling can operate independently of gravity, such as by capillary action or pressure drop through a valve. The vaporizer 300 can be configured to mix in varying proportions the fluids contained in the container 202a and the container 202b and vaporize the mixture at controlled rates in response to mechanical input from a component of the vapor device 100, and/or in response to control signals from the processor 102 or another component. In an aspect, a mixing element 302 can be coupled to the container 202a and the container 202b. The mixing element can, in response to a control signal from the processor 102, withdraw select quantities of vaporizable material in order to create a customized mixture of different types of vaporizable material. Vaporizable material (e.g., fluid) can be supplied by one or more replaceable cartridges 206a and 206b. The one or more replaceable cartridges 206a and 206b can contain a vaporizable material. If the vaporizable material is liquid, the cartridge can comprise the wick 204a or 204b to aid in transporting the liquid to a mixing chamber 208. In the alternative, some other transport mode can be used. Each of the one or more replaceable cartridges 206a and 206b can be configured to fit inside and engage removably with a receptacle (such as the container 202a or the container 202b and/or a secondary container) of the vapor device 100. In an alternative, or in addition, one or more fluid containers 210a and 210b can be fixed in the vapor device 100 and configured to be refillable. In an aspect, one or more materials can be vaporized at a single time by the vaporizer 300. For example, some material can be vaporized and drawn through an exhaust port 212 and/or some material can be vaporized and exhausted via a smoke simulator outlet (not shown).

In an aspect, the vaporizer 300 can comprise a pressure sensor 218 configured for measuring a volume and/or rate of air inspired and/or expired by a user's lungs. In an aspect, the pressure sensor 218 can be configured to permit the vaporizer 200 to function as a spirometer. The user can exhale into the vaporizer 200 through the outlet 212. The pressure sensor 218 can be coupled to the outlet 212 and measure the volume and/or rate of the exhalation. In another aspect, the user can inhale from the vaporizer 200 through the outlet 212. The pressure sensor 218 can be coupled to the outlet 212 to measure the volume and/or rate of the inspiration. In an aspect, the pressure sensor 218 can be configured to calculate the air flow from a pressure difference measured across an obstruction in a flow channel extending from the outlet 212 an air intake. During a user expiration and/or inspiration, electrical signals from the pressure sensor 218 can be amplified, converted from analog to digital by an A/D converter, and stored in a memory device.

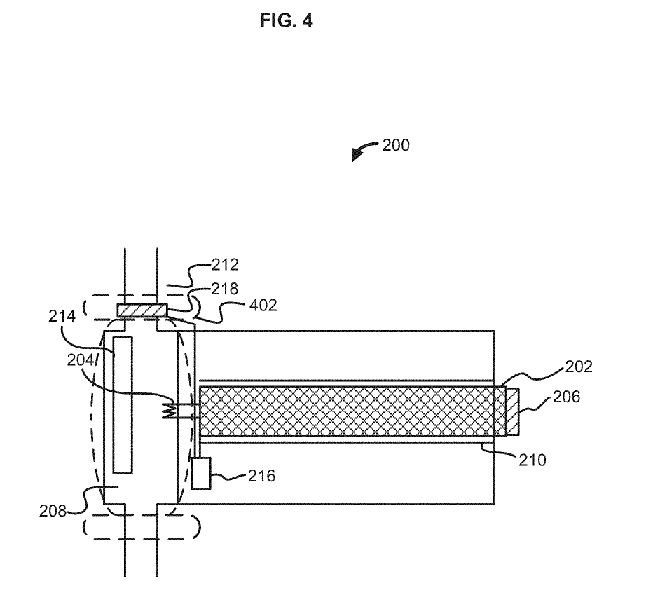

FIG. 4 illustrates a vaporizer 200 that comprises the elements of the vaporizer 200 with a heating casing 402. The heating casing 402 can enclose the heating element 214 or can be adjacent to the heating element 214. The heating casing 402 is illustrated with dashed lines, indicating components contained therein. The heating casing 402 can be made of ceramic, metal, and/or porcelain. The heating casing 402 can have varying thickness. In an aspect, the heating casing 402 can be coupled to the multiplexer 216 to receive power to heat the heating casing 402. In another aspect, the heating casing 402 can be coupled to the heating element 214 to heat the heating casing 402. In another aspect, the heating casing 402 can serve an insulation role.

In an aspect, the vaporizer 200 can comprise a pressure sensor 218 configured for measuring a volume and/or rate of air inspired and/or expired by a user's lungs. In an aspect, the pressure sensor 218 can be configured to permit the vaporizer 200 to function as a spirometer. The user can exhale into the vaporizer 200 through the outlet 212. The pressure sensor 218 can be coupled to the outlet 212 and measure the volume and/or rate of the exhalation. In another aspect, the user can inhale from the vaporizer 200 through the outlet 212. The pressure sensor 218 can be coupled to the outlet 212 to measure the volume and/or rate of the inspiration. In an aspect, the pressure sensor 218 can be configured to calculate the air flow from a pressure difference measured across an obstruction in a flow channel extending from the outlet 212 an air intake. During a user expiration and/or inspiration, electrical signals from the pressure sensor 218 can be amplified, converted from analog to digital by an A/D converter, and stored in a memory device.

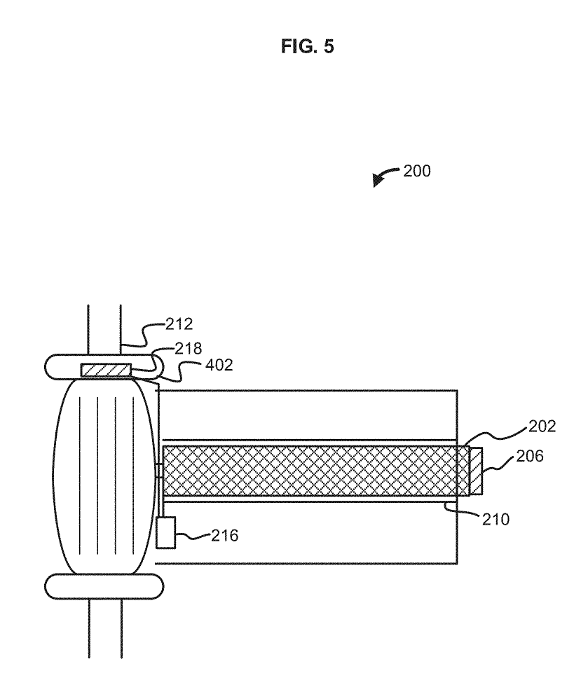

FIG. 5 illustrates the vaporizer 200 of FIG. 2 and FIG. 4, but illustrates the heating casing 402 with solid lines, indicating components contained therein. Other placements of the heating casing 402 are contemplated. For example, the heating casing 402 can be placed after the heating element 214 and/or the mixing chamber 208.

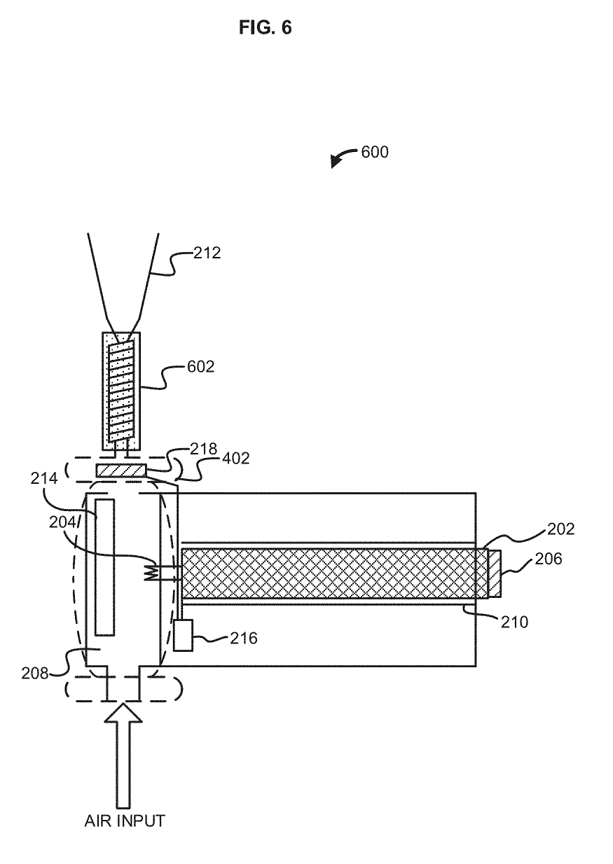

FIG. 6 illustrates a vaporizer 600 that comprises the elements of the vaporizer 200 of FIG. 2 and FIG. 4, with the addition of a cooling element 602. The vaporizer 600 can optionally comprise the heating casing 402. The cooling element 602 can comprise one or more of a powered cooling element, a cooling air system, and/or or a cooling fluid system. The cooling element 602 can be self-powered, co-powered, or directly powered by a battery and/or charging system within the vapor device 100 (e.g., the power supply 120). In an aspect, the cooling element 602 can comprise an electrically connected conductive coil, grating, and/or other design to efficiently distribute cooling to the at least one of the vaporized and/or non-vaporized air. For example, the cooling element 602 can be configured to cool air as it is brought into the vaporizer 600/mixing chamber 208 and/or to cool vapor after it exits the mixing chamber 208. The cooling element 602 can be deployed such that the cooling element 602 is surrounded by the heated casing 402 and/or the heating element 214. In another aspect, the heated casing 402 and/or the heating element 214 can be surrounded by the cooling element 602. The cooling element 602 can utilize at least one of cooled air, cooled liquid, and/or cooled matter.

In an aspect, the cooling element 602 can be a coil of any suitable length and can reside proximate to the inhalation point of the vapor (e.g., the exhaust port 212). The temperature of the air is reduced as it travels through the cooling element 602. In an aspect, the cooling element 602 can comprise any structure that accomplishes a cooling effect. For example, the cooling element 602 can be replaced with a screen with a mesh or grid-like structure, a conical structure, and/or a series of cooling airlocks, either stationary or opening, in a periscopic/telescopic manner. The cooling element 602 can be any shape and/or can take multiple forms capable of cooling heated air, which passes through its space.

In an aspect, the cooling element 602 can be any suitable cooling system for use in a vapor device. For example, a fan, a heat sink, a liquid cooling system, a chemical cooling system, combinations thereof, and the like. In an aspect, the cooling element 602 can comprise a liquid cooling system whereby a fluid (e.g., water) passes through pipes in the vaporizer 600. As this fluid passes around the cooling element 602, the fluid absorbs heat, cooling air in the cooling element 602. After the fluid absorbs the heat, the fluid can pass through a heat exchanger which transfers the heat from the fluid to air blowing through the heat exchanger. By way of further example, the cooling element 602 can comprise a chemical cooling system that utilizes an endothermic reaction. An example of an endothermic reaction is dissolving ammonium nitrate in water. Such endothermic process is used in instant cold packs. These cold packs have a strong outer plastic layer that holds a bag of water and a chemical, or mixture of chemicals, that result in an endothermic reaction when dissolved in water. When the cold pack is squeezed, the inner bag of water breaks and the water mixes with the chemicals. The cold pack starts to cool as soon as the inner bag is broken, and stays cold for over an hour. Many instant cold packs contain ammonium nitrate. When ammonium nitrate is dissolved in water, it splits into positive ammonium ions and negative nitrate ions. In the process of dissolving, the water molecules contribute energy, and as a result, the water cools down. Thus, the vaporizer 600 can comprise a chamber for receiving the cooling element 602 in the form of a “cold pack.” The cold pack can be activated prior to insertion into the vaporizer 600 or can be activated after insertion through use of a button/switch and the like to mechanically activate the cold pack inside the vaporizer 400.

In an aspect, the cooling element 602 can be selectively moved within the vaporizer 600 to control the temperature of the air mixing with vapor. For example, the cooling element 602 can be moved closer to the exhaust port 212 or further from the exhaust port 212 to regulate temperature. In another aspect, insulation can be incorporated as needed to maintain the integrity of heating and cooling, as well as absorbing any unwanted condensation due to internal or external conditions, or a combination thereof. The insulation can also be selectively moved within the vaporizer 600 to control the temperature of the air mixing with vapor. For example, the insulation can be moved to cover a portion, none, or all of the cooling element 602 to regulate temperature.

In an aspect, the vaporizer 600 can comprise a pressure sensor 218 configured for measuring a volume and/or rate of air inspired and/or expired by a user's lungs. The pressure sensor 218 can be located on either side of the cooling element 602. In an aspect, the pressure sensor 218 can be configured to permit the vaporizer 600 to function as a spirometer. The user can exhale into the vaporizer 600 through the outlet 212. The pressure sensor 218 can be coupled to the outlet 212 and measure the volume and/or rate of the exhalation. In another aspect, the user can inhale from the vaporizer 600 through the outlet 212. The pressure sensor 218 can be coupled to the outlet 212 to measure the volume and/or rate of the inspiration. In an aspect, the pressure sensor 218 can be configured to calculate the air flow from a pressure difference measured across an obstruction in a flow channel extending from the outlet 212 an air intake. During a user expiration and/or inspiration, electrical signals from the pressure sensor 218 can be amplified, converted from analog to digital by an A/D converter, and stored in a memory device.

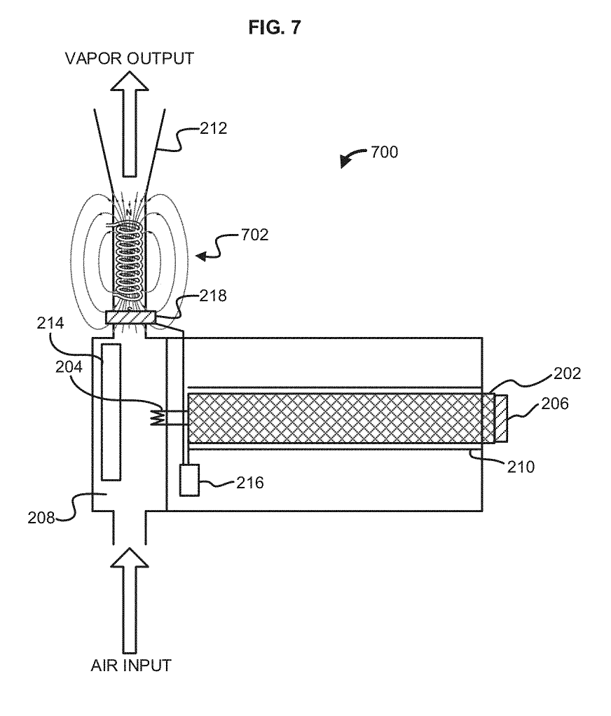

FIG. 7 illustrates a vaporizer 700 that comprises elements in common with the vaporizer 200. The vaporizer 700 can optionally comprise the heating casing 402 (not shown) and/or the cooling element 602 (not shown). The vaporizer 700 can comprise a magnetic element 702. The magnetic element 702 can apply a magnetic field to vapor after exiting the mixing chamber 208. The magnetic field can cause positively and negatively charged particles in the vapor to curve in opposite directions, according to the Lorentz force law with two particles of opposite charge. The magnetic field can be created by at least one of an electric current generating a charge or a pre-charged magnetic material deployed within the vapor device 100. In an aspect, the magnetic element 702 can be built into the mixing chamber 208, the cooling element 602, the heating casing 402, or can be a separate magnetic element 702.

In an aspect, the vaporizer 700 can comprise a pressure sensor 218 configured for measuring a volume and/or rate of air inspired and/or expired by a user's lungs. The pressure sensor 218 can be located on either side of the magnetic element 702. In an aspect, the pressure sensor 218 can be configured to permit the vaporizer 700 to function as a spirometer. The user can exhale into the vaporizer 700 through the outlet 212. The pressure sensor 218 can be coupled to the outlet 212 and measure the volume and/or rate of the exhalation. In another aspect, the user can inhale from the vaporizer 700 through the outlet 212. The pressure sensor 218 can be coupled to the outlet 212 to measure the volume and/or rate of the inspiration. In an aspect, the pressure sensor 218 can be configured to calculate the air flow from a pressure difference measured across an obstruction in a flow channel extending from the outlet 212 an air intake. During a user expiration and/or inspiration, electrical signals from the pressure sensor 218 can be amplified, converted from analog to digital by an A/D converter, and stored in a memory device.

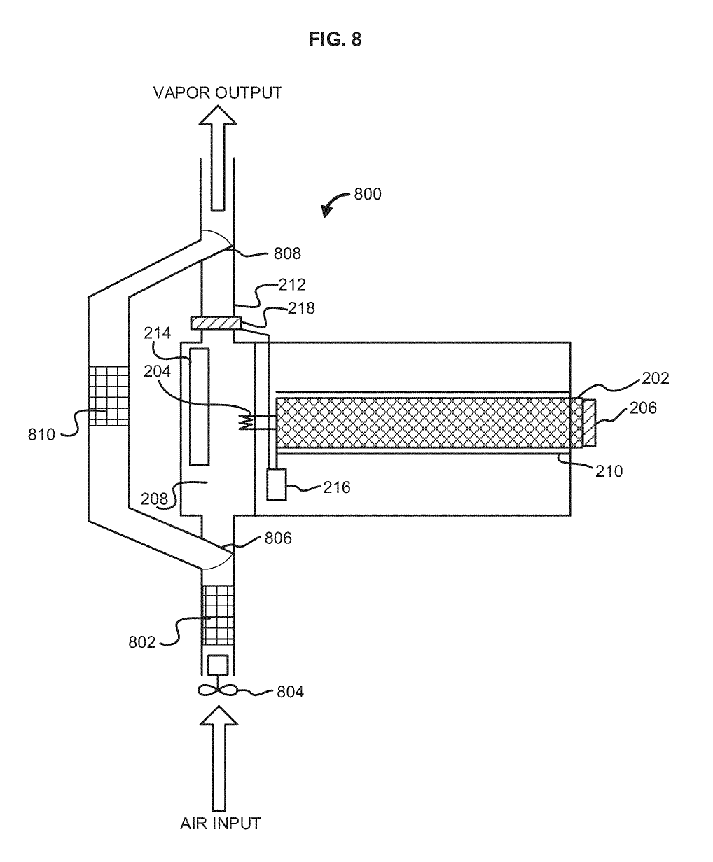

FIG. 8 illustrates a vaporizer 800 that comprises elements in common with the vaporizer 200. In an aspect, the vaporizer 800 can comprise a filtration element 802. The filtration element 802 can be configured to remove (e.g., filter, purify, etc) contaminants from air entering the vaporizer 800. The filtration element 802 can optionally comprise a fan 804 to assist in delivering air to the filtration element 802. The vaporizer 800 can be configured to intake air into the filtration element 802, filter the air, and pass the filtered air to the mixing chamber 208 for use in vaporizing the one or more vaporizable or non-vaporizable materials. In another aspect, the vaporizer 800 can be configured to intake air into the filtration element 802, filter the air, and bypass the mixing chamber 208 by engaging a door 806 and a door 808 to pass the filtered air directly to the exhaust port 212 for inhalation by a user. In an aspect, filtered air that bypasses the mixing chamber 208 by engaging the door 806 and the door 808 can pass through a second filtration element 810 to further remove (e.g., filter, purify, etc) contaminants from air entering the vaporizer 800. In an aspect, the vaporizer 800 can be configured to deploy and/or mix a proper/safe amount of oxygen which can be delivered either via the one or more replaceable cartridges 206 or via air pumped into a mask from external air and filtered through the filtration element 802 and/or the filtration element 810.

In an aspect, the filtration element 802 and/or the filtration element 810 can comprise cotton, polymer, wool, satin, meta materials and the like. The filtration element 802 and/or the filtration element 810 can comprise a filter material that at least one airborne particle and/or undesired gas by a mechanical mechanism, an electrical mechanism, and/or a chemical mechanism. The filter material can comprise one or more pieces of, a filter fabric that can filter out one or more airborne particles and/or gasses. The filter fabric can be a woven and/or non-woven material. The filter fabric can be made from natural fibers (e.g., cotton, wool, etc.) and/or from synthetic fibers (e.g., polyester, nylon, polypropylene, etc.). The thickness of the filter fabric can be varied depending on the desired filter efficiencies and/or the region of the apparel where the filter fabric is to be used. The filter fabric can be designed to filter airborne particles and/or gasses by mechanical mechanisms (e.g., weave density), by electrical mechanisms (e.g., charged fibers, charged metals, etc.), and/or by chemical mechanisms (e.g., absorptive charcoal particles, adsorptive materials, etc.). In as aspect, the filter material can comprise electrically charged fibers such as, but not limited to, FILTRETE by 3M. In another aspect, the filter material can comprise a high density material similar to material used for medical masks which are used by medical personnel in doctors' offices, hospitals, and the like. In an aspect, the filter material can be treated with an anti-bacterial solution and/or otherwise made from anti-bacterial materials. In another aspect, the filtration element 802 and/or the filtration element 810 can comprise electrostatic plates, ultraviolet light, a HEPA filter, combinations thereof; and the like.

In an aspect, the vaporizer 800 can comprise a pressure sensor 218 configured for measuring a volume and/or rate of air inspired and/or expired by a user's lungs. The pressure sensor 218 can be located before or after the filtration element 810. In an aspect, the pressure sensor 218 can be configured to permit the vaporizer 800 to function as a spirometer. The user can exhale into the vaporizer 800 through the outlet 212. The pressure sensor 218 can be coupled to the outlet 212 and measure the volume and/or rate of the exhalation. In another aspect, the user can inhale from the vaporizer 800 through the outlet 212. The pressure sensor 218 can be coupled to the outlet 21.2 to measure the volume and/or rate of the inspiration. In an aspect, the pressure sensor 218 can be configured to calculate the air flow from a pressure difference measured across an obstruction in a flow channel extending from the outlet 212 an air intake. During a user expiration and/or inspiration, electrical signals from the pressure sensor 218 can be amplified, converted from analog to digital by an A/D converter, and stored in a memory device.

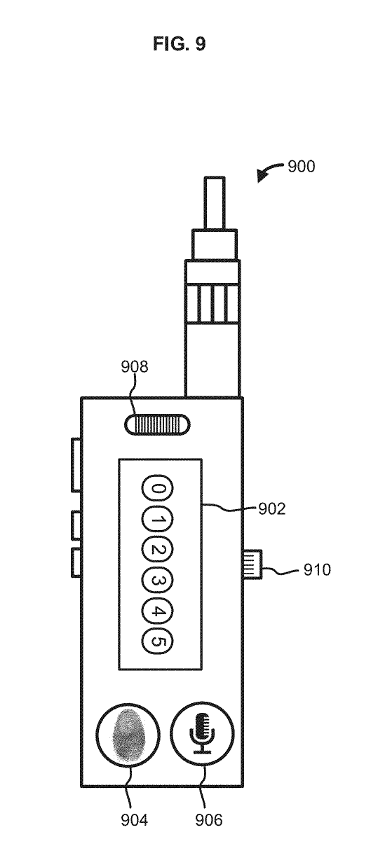

FIG. 9 illustrates an exemplary vapor device 900. The exemplary vapor device 900 can comprise the vapor device 100 and/or any of the vaporizers disclosed herein. The exemplary vapor device 900 illustrates a display 902. The display 902 can be a touchscreen. The display 902 can be configured to enable a user to control any and/or all functionality of the exemplary vapor device 900. For example, a user can utilize the display 902 to enter a pass code to lock and/or unlock the exemplary vapor device 900. The exemplary vapor device 900 can comprise a biometric interface 904. For example, the biometric interface 904 can comprise a fingerprint scanner, an eye scanner, a facial scanner, and the like. The biometric interface 904 can be configured to enable a user to control any and/or all functionality of the exemplary vapor device 900. The exemplary vapor device 900 can comprise an audio interface 906. The audio interface 906 can comprise a button that, when engaged, enables a microphone 908. The microphone 908 can receive audio signals and provide the audio signals to a processor for interpretation into one or more commands to control one or more functions of the exemplary vapor device 900. The exemplary vapor device 900 can comprise an input/output port 910. The input/output port 910 can adhere to any proprietary standard. In another aspect, the input/output port 910 can comprise one or more of, a USB connection, a dock connector (e.g., 20-24-30 pin connectors, lightning port connection, etc), Portable Digital Media Interface, and the like. The input/output port 910 can be used to pass power and/or data between an electronic communication device and the exemplary vapor device 900.

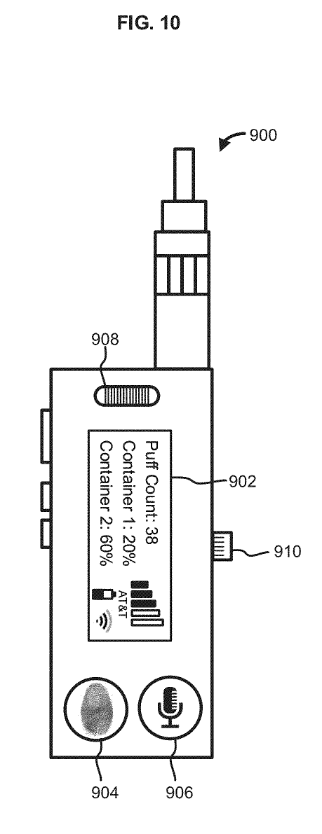

FIG. 10 illustrates exemplary information that can be provided to a user via the display 902 of the exemplary vapor device 900. The display 902 can provide information to a user such as a puff count, an amount of vaporizable material remaining in one or more containers, battery remaining, signal strength, combinations thereof, and the like.

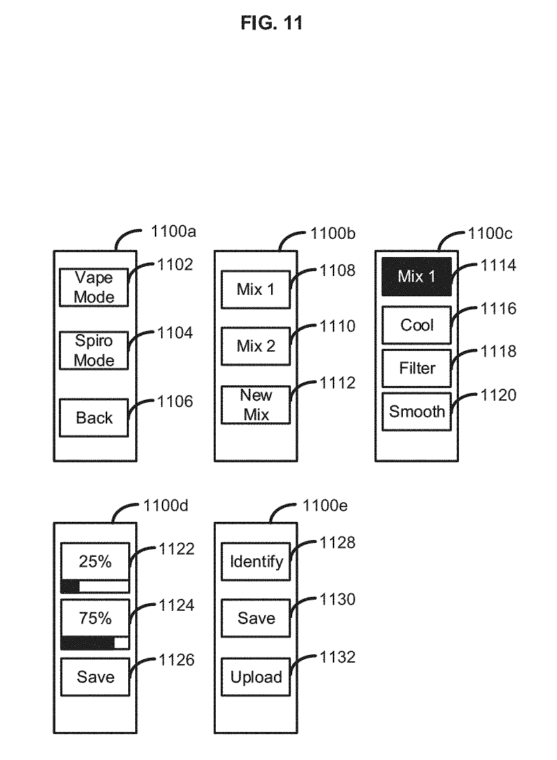

FIG. 11 illustrates a series of user interfaces that can be provided via the display 902 of the exemplary vapor device 900. In an aspect, the exemplary vapor device 900 can be configured for one or more of multi-mode vapor usage. For example, the exemplary vapor device 900 can be configured to enable a user to inhale vapor (nape mode) or to perform spirometer functions, including inhale/release vapor comprising respiratory medication (spirometer mode). User interface 1100a provides a user with interface elements to select which mode the user wishes to engage, a Vape Mode 1102, a Spirometer (Spiro) Mode 1104, or an option to go back 1106 and return to the previous screen. The interface element Vape Mode 1102 enables a user to engage a vaporizer to generate a vapor for inhalation. The interface element Spirometer Mode 1104 enables a user to engage the vaporizer to activate a spirometer functionality and to generate a vapor made up of respiratory medication for inhalation and/or release into the atmosphere.

In the event a user selects the Vape Mode 1102, the exemplary vapor device 900 will be configured to vaporize material and provide the resulting vapor to the user for inhalation. The user can be presented with user interface 1100b which provides the user an option to select interface elements that will determine which vaporizable material to vaporize. For example, an option of Mix 1 1108, Mix 2 1110, or a New Mix 1112. The interface element Mix 1 1108 enables a user to engage one or more containers that contain vaporizable material in a predefined amount and/or ratio. In an aspect, a selection of Mix 1 1108 can result in the exemplary vapor device 900 engaging a single container containing a single type of vaporizable material or engaging a plurality of containers containing a different types of vaporizable material in varying amounts. The interface element Mix 2 1110 enables a user to engage one or more containers that contain vaporizable material in a predefined amount and/or ratio. In an aspect, a selection of Mix 2 1110 can result in the exemplary vapor device 900 engaging a single container containing a single type of vaporizable material or engaging a plurality of containers containing a different types of vaporizable material in varying amounts. In an aspect, a selection of New Mix 1112 can result in the exemplary vapor device 900 receiving a new mixture, formula, recipe, etc . . . of vaporizable materials and/or engage one or more containers that contain vaporizable material in the new mixture.

Upon selecting, for example, the Mix 1 1108, the user can be presented with user interface 1100c. User interface 1100c indicates to the user that Mix 1 has been selected via an indicator 1114. The user can be presented with options that control how the user wishes to experience the selected vapor. The user can be presented with interface elements Cool 1116, Filter 1118, and Smooth 1120. The interface element Cool 1116 enables a user to engage one or more cooling elements to reduce the temperature of the vapor. The interface element Filter 1118 enables a user to engage one or more filter elements to filter the air used in the vaporization process. The interface element Smooth 1120 enables a user to engage one or more heating casings, cooling elements, filter elements, and/or magnetic elements to provide the user with a smoother vaping experience.

Upon selecting New Mix 1112, the user can be presented with user interface 1100d. User interface 1100d provides the user with a container one ratio interface element 1122, a container two ratio interface element 1124, and Save 1126. The container one ratio interface element 1122 and the container two ratio interface element 1124 provide a user the ability to select an amount of each type of vaporizable material contained in container one and/or container two to utilize as a new mix. The container one ratio interface element 1122 and the container two ratio interface element 1124 can provide a user with a slider that adjusts the percentages of each type of vaporizable material based on the user dragging the slider. In an aspect, a mix can comprise 100% on one type of vaporizable material or any percent combination (e.g., 50/50, 75/25, 85/15, 95/5, etc. . . . ). Once the user is satisfied with the new mix, the user can select Save 1126 to save the new mix for later use.

In the event a user selects the Spirometer Mode 1104, the exemplary vapor device 900 will be configured to measure a flow, volume, and/or rate of inhalation/exhalation by the user. The exemplary vapor device 900 can ultimately vaporize material comprising respiratory medication and release the resulting vapor into the atmosphere and/or for direct user inhalation based on an analysis of data generated by the spirometer function. The user can be presented with user interface 1100b, 1100c, and/or 1100d as described above, but the resulting vapor will comprise respiratory medication.

In an aspect, the user can be presented with user interface 1100e. The user interface 1100e can provide the user with interface elements Identify 1128, Save 1130, and Upload 1132. The interface element Identify 1128 enables a user to engage one or more sensors in the exemplary vapor device 900 to analyze the surrounding environment. For example, activating the interface element Identify 1128 can engage a sensor to determine the presence of a negative environmental condition such as smoke, a bad smell, chemicals, etc. Activating the interface element Identify 1128 can engage a sensor to determine the presence of a positive environmental condition, for example, an aroma. The interface element Save 1130 enables a user to save data related to the analyzed negative and/or positive environmental condition in memory local to the exemplary vapor device 900. The interface element Upload 1132 enables a user to engage a network access device to transmit data related to the analyzed negative and/or positive environmental condition to a remote server for storage and/or analysis,

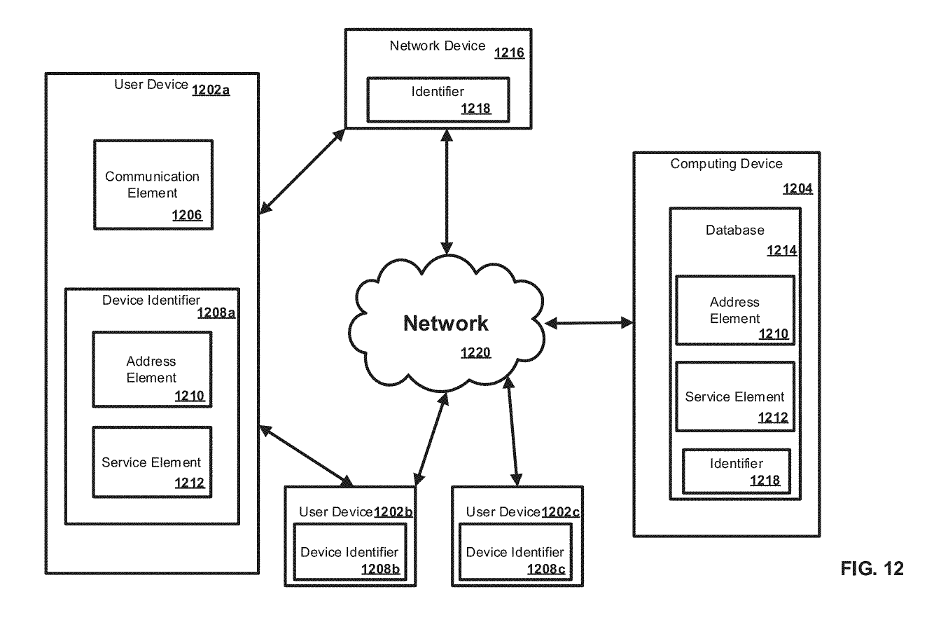

In one aspect of the disclosure, a system can be configured to provide services such as network-related services to a user device. FIG. 12 illustrates various aspects of an exemplary environment in which the present methods and systems can operate. The present disclosure is relevant to systems and methods for providing services to a user device, for example, electronic vapor devices which can include, but are not limited to, a vape-bot, micro-vapor device, vapor pipe, e-cigarette, hybrid handset and vapor device, and the like. Other user devices that can be used in the systems and methods include, but are not limited to, a smart watch (and any other form of “smart” wearable technology), a smartphone, a tablet, a laptop, a desktop, and the like. In an aspect, one or more network devices can be configured to provide various services to one or more devices, such as devices located at or near a premises. In another aspect, the network devices can be configured to recognize an authoritative device for the premises and/or a particular service or services available at the premises. As an example, an authoritative device can be configured to govern or enable connectivity to a network such as the Internet or other remote resources, provide address and/or configuration services like DHCP, and/or provide naming or service discovery services for a premises, or a combination thereof. Those skilled in the art will appreciate that present methods may be used in various types of networks and systems that employ both digital and analog equipment. One skilled in the art will appreciate that provided herein is a functional description and that the respective functions can be performed by software, hardware, or a combination of software and hardware.

The network and system can comprise a user device 1202a, 1202b, and/or 1202c in communication with a computing device 1204 such as a server, for example. The computing device 1204 can be disposed locally or remotely relative to the user device 1202a, 1202b, and/or 1202c. As an example, the user device 1202a, 1202b, and/or 1202c and the computing device 1204 can be in communication via a private and/or public network 1220 such as the Internet or a local area network. Other forms of communications can be used such as wired and wireless telecommunication channels, for example. In another aspect, the user device 1202a, 1202b, and/or 1202c can communicate directly without the use of the network 1220 (for example, via Bluetooth®, infrared, and the like).

In an aspect, the user device 1202a, 1202b, and/or 1202c can be an electronic device such as an electronic vapor device (e.g., vape-bot, micro-vapor device, vapor pipe, e-cigarette, hybrid handset and vapor device), a smartphone, a smart watch, a computer, a smartphone, a laptop, a tablet, a set top box, a display device, or other device capable of communicating with the computing device 1204. As an example, the user device 1202a, 1202b, and/or 1202c can comprise a communication element 1206 for providing an interface to a user to interact with the user device 1202a, 1202b, and/or 1202c and/or the computing device 1204. The communication element 1206 can be any interface for presenting and/or receiving information to/from the user, such as user feedback. An example interface may be communication interface such as a web browser (e.g., Internet Explorer, Mozilla Firefox, Google Chrome, Safari, or the like). Other software, hardware, and/or interfaces can be used to provide communication between the user and one or more of the user device 1202a, 1202b, and/or 1202c and the computing device 1204. In an aspect, the user device 1202a, 1202b, and/or 1202c can have at least one similar interface quality such as a symbol, a voice activation protocol, a graphical coherence, a startup sequence continuity element of sound, light, vibration or symbol. In an aspect, the interface can comprise at least one of lighted signal lights, gauges, boxes, forms, words, video, audio scrolling, user selection systems, vibrations, check marks, avatars, matrix, visual images, graphic designs, lists, active calibrations or calculations, 2D interactive fractal designs, 3D fractal designs, 2D and/or 3D representations of vapor devices and other interface system functions.

As an example, the communication element 1206 can request or query various files from a local source and/or a remote source. As a further example, the communication element 1206 can transmit data to a local or remote device such as the computing device 1204.

In an aspect, the user device 1202a, 1202b, and/or 1202c can be associated with a user identifier or device identifier 1208a, 1208b, and/or 1208c. As an example, the device identifier 1208a, 1208b, and/or 1208c can be any identifier, token, character, string, or the like, for differentiating one user or user device (e.g., user device 1202a, 1202b, and/or 1202c) from another user or user device. In a further aspect, the device identifier 1208a, 1208b, and/or 1208c can identify a user or user device as belonging to a particular class of users or user devices. As a further example, the device identifier 1208a, 1208b, and/or 1208c can comprise information relating to the user device such as a manufacturer, a model or type of device, a service provider associated with the user device 1202a, 1202b, and/or 1202c, a state of the user device 1202a, 1202b, and/or 1202c, a locator, and/or a label or classifier. Other information can be represented by the device identifier 1208a, 1208b, and/or 1208c.

In an aspect, the device identifier 1208a, 1208b, and/or 1208c can comprise an address element 1210 and a service element 1212. In an aspect, the address element 1210 can comprise or provide an internet protocol address, a network address, a media access control (MAC) address, an Internet address, or the like. As an example, the address element 1210 can be relied upon to establish a communication session between the user device 1202a, 1202b, and/or 1202c and the computing device 1204 or other devices and/or networks. As a further example, the address element 1210 can be used as an identifier or locator of the user device 1202a, 1202b, and/or 1202c. In an aspect, the address element 1210 can be persistent for a particular network.

In an aspect, the service element 1212 can comprise an identification of a service provider associated with the user device 1202a, 1202b, and/or 1202c and/or with the class of user device 1202a, 1202b, and/or 1202c. The class of the user device 1202a, 1202b, and/or 1202c can be related to a type of device, capability of device, type of service being provided, and/or a level of service. As an example, the service element 1212 can comprise information relating to or provided by a communication service provider (e.g., Internet service provider) that is providing or enabling data flow such as communication services to and/or between the user device 1202a, 1202b, and/or 1202c. As a further example, the service element 1212 can comprise information relating to a preferred service provider for one or more particular services relating to the user device 1202a, 1202b, and/or 1202c. In an aspect, the address element 1210 can be used to identify or retrieve data from the service element 1212, or vice versa. As a further example, one or more of the address element 1210 and the service element 1212 can be stored remotely from the user device 1202a, 1202b, and/or 1202c and retrieved by one or more devices such as the user device 1202a, 1202b, and/or 1202c and the computing device 1204. Other information can be represented by the service element 1212.

In an aspect, the computing device 1204 can be a server for communicating with the user device 1202a, 1202b, and/or 1202c. As an example, the computing device 1204 can communicate with the user device 1202a, 1202b, and/or 1202c for providing data and/or services. As an example, the computing device 1204 can provide services such as data sharing, data syncing, network (e.g., Internet) connectivity, network printing, media management (e.g., media server), content services, streaming services, broadband services, or other network-related services. In an aspect, the computing device 1204 can allow the user device 1202a, 1202b, and/or 1202c to interact with remote resources such as data, devices, and files. As an example, the computing device can be configured as (or disposed at) a central location, which can receive content (e.g., data) from multiple sources, for example, user devices 1202a, 1202b, and/or 1202c. The computing device 1204 can combine the content from the multiple sources and can distribute the content to user (e.g., subscriber) locations via a distribution system.

In an aspect, one or more network devices 1216 can be in communication with a network such as network 1220. As an example, one or more of the network devices 1216 can facilitate the connection of a device, such as user device 1202a, 1202b, and/or 1202c, to the network 1220. As a further example, one or more of the network devices 1216 can be configured as a wireless access point (WAP). In an aspect, one or more network devices 1216 can be configured to allow one or more wireless devices to connect to a wired and/or wireless network using Wi-Fi, Bluetooth or any desired method or standard.

In an aspect, the network devices 1216 can be configured as a local area network (LAN). As an example, one or more network devices 1216 can comprise a dual band wireless access point. As an example, the network devices 1216 can be configured with a first service set identifier (SSID) (e.g., associated with a user network or private network) to function as a local network for a particular user or users. As a further example, the network devices 1216 can be configured with a second service set identifier (SSID) (e.g., associated with a public/community network or a hidden network) to function as a secondary network or redundant network for connected communication devices.

In an aspect, one or more network devices 1216 can comprise an identifier 1218. As an example, one or more identifiers can be or relate to an Internet Protocol (IP) Address IPV4/IPV6 or a media access control address (MAC address) or the like. As a further example, one or more identifiers 1218 can be a unique identifier for facilitating communications on the physical network segment. In an aspect, each of the network devices 1216 can comprise a distinct identifier 1218. As an example, the identifiers 1218 can be associated with a physical location of the network devices 1216.

In an aspect, the computing device 1204 can manage the communication between the user device 1202a, 1202b, and/or 1202c and a database 1214 for sending and receiving data therebetween. As an example, the database 1214 can store a plurality of files (e.g., web pages), user identifiers or records, or other information. In one aspect, the database 1214 can store user device 1202a, 1202b, and/or 1202c usage information (including chronological usage), type of vaporizable and/or non-vaporizable material used, frequency of usage, location of usage, recommendations, communications (e.g., text messages, advertisements, photo messages), simultaneous use of multiple devices, and the like). The database 1214 can collect and store data to support cohesive use, wherein cohesive use is indicative of the use of a first electronic vapor devices and then a second electronic vapor device is synced chronologically and logically to provide the proper specific properties and amount of vapor based upon a designed usage cycle. As a further example, the user device 1202a, 1202b, and/or 1202c can request and/or retrieve a file front the database 1214. The user device 1202a, 1202b, and/or 1202c can thus sync locally stored data with more current data available from the database 1214. Such syncing can be set to occur automatically on a set time schedule, on demand, and/or in real-time. The computing device 1204 can be configured to control syncing functionality. For example, a user can select one or more of the user device 1202a, 1202b, and/or 1202c to never by synced, to be the master data source for syncing, and the like. Such functionality can be configured to be controlled by a master user and any other user authorized by the master user or agreement.

In an aspect, data can be derived by system and/or device analysis. Such analysis can comprise at least by one of instant analysis performed by the user device 1202a, 1202b, and/or 1202c or archival data transmitted to a third party for analysis and returned to the user device 1202a, 1202b, and/or 1202c and/or computing device 1204. The result of either data analysis can be communicated to a user of the user device 1202a, 1202b, and/or 1202c to, for example, inform the user of their eVapor use and/or lifestyle options. In an aspect, a result can be transmitted back to at least one authorized user interface. In an aspect, the analysis can comprise an analysis of data generated by use of the user device 1202a, 1202b, and/or 1202c as a spirometer.

In an aspect, the database 1214 can store information relating to the user device 1202a, 1202b, and/or 1202c such as the address element 1210 and/or the service element 1212. As an example, the computing device 1204 can obtain the device identifier 1208a, 1208b, and/or 1208c from the user device 1202a, 1202b, and/or 1202c and retrieve information from the database 1214 such as the address element 1210 and/or the service elements 1212. As a further example, the computing device 1204 can obtain the address element 1210 from the user device 1202a, 1202b, and/or 1202c and can retrieve the service element 1212 from the database 1214, or vice versa. Any information can be stored in and retrieved from the database 1214. The database 1214 can be disposed remotely from the computing device 1204 and accessed via direct or indirect connection. The database 1214 can be integrated with the computing device 1204 or some other device or system.

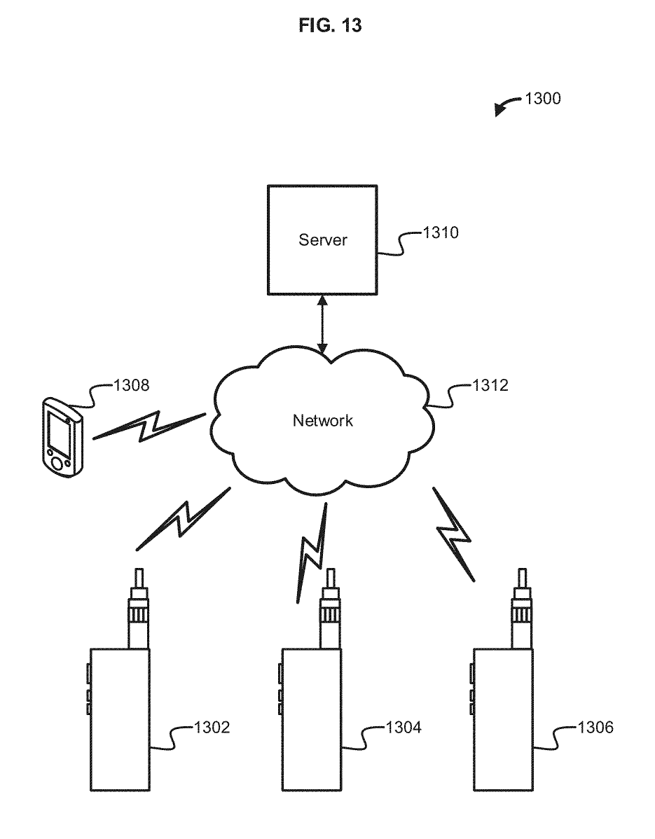

FIG. 13 illustrates an ecosystem 1300 configured for sharing and/or syncing data such as respiratory medication recommendations, usage information (including chronological usage), type of vaporizable and/or non-vaporizable material used, frequency of usage, location of usage, recommendations, communications (e.g., text messages, advertisements, photo messages), simultaneous use of multiple devices, and the like) between one or more devices such as a vapor device 1302, a vapor device 1304, a vapor device 1306, and an electronic communication device 1308. In an aspect, the vapor device 1302, the vapor device 1304, the vapor device 1306 can be one or more of an e-cigarette, an e-cigar, an electronic vapor modified device, a hybrid electronic communication handset coupled/integrated vapor device, a micro-sized electronic vapor device, or a robotic vapor device. In an aspect, the electronic communication device 1308 can comprise one or more of a smartphone, a smart watch, a tablet, a laptop, and the like.

In an aspect data generated, gathered, created, etc., by one or more of the vapor device 1302, the vapor device 1304, the vapor device 1306, and/or the electronic communication device 1308 can be uploaded to and/or downloaded from a central server 1310 via a network 1312, such as the Internet. Such uploading and/or downloading can be performed via any form of communication including wired and/or wireless. In an aspect, the vapor device 1302, the vapor device 1304, the vapor device 1306, and/or the electronic communication device 1308 can be configured to communicate via cellular communication, WiFi communication, Bluetooth® communication, satellite communication, and the like. The central server 1310 can store uploaded data and associate the uploaded data with a user and/or device that uploaded the data. The central server 1310 can access unified account and tracking information to determine devices that are associated with each other, for example devices that are owned/used by the same user. The central server 1310 can utilize the unified account and tracking information to determine which of the vapor device 1302, the vapor device 1304, the vapor device 1306, and/or the electronic communication device 1308, if any, should receive data uploaded to the central server 1310.

For example, the vapor device 1302 can be configured to upload usage information related to vaporizable material consumed and the electronic communication device 1308 can be configured to upload location information related to location of the vapor device 1302. The central server 1310 can receive both the usage information and the location information, access the unified account and tracking information to determine that both the vapor device 1302 and the electronic communication device 1308 are associated with the same user. The central server 1310 can thus correlate the user's location along with the type, amount, and/or timing of usage of the vaporizable material. The central server 1310 can further determine which of the other devices are permitted to receive such information and transmit the information based on the determined permissions. In an aspect, the central server 1310 can transmit the correlated information to the electronic communication device 1308 which can then subsequently use the correlated information to recommend a specific type of vaporizable material to the user when the user is located in the same geographic position indicated by the location information.

In another aspect, the central server 1310 can provide one or more social networking services for users of the vapor device 1302, the vapor device 1304, the vapor device 1306, and/or the electronic communication device 1308. Such social networking services include, but are not limited to, messaging (e.g. text, image, and/or video), mixture sharing, product recommendations, location sharing, product ordering, and the like.

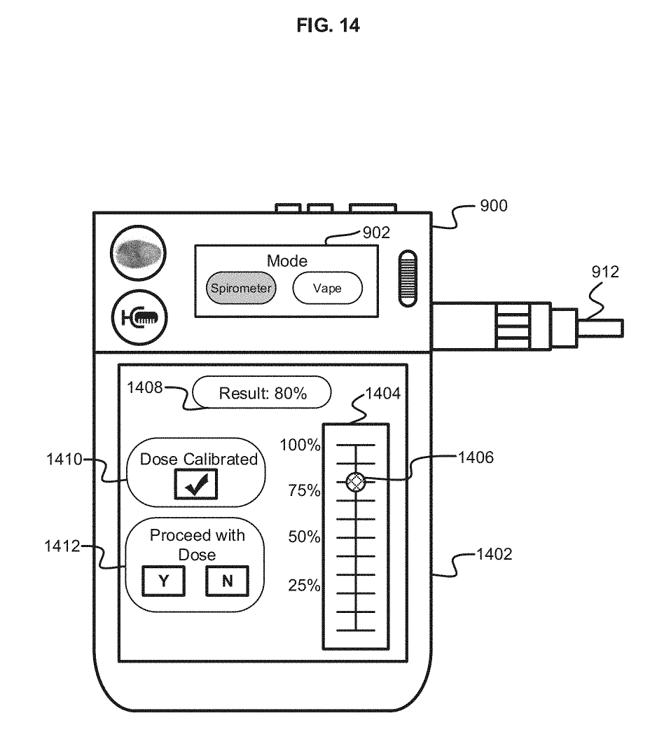

In an aspect, illustrated in FIG. 14, provided is an exemplary vapor device 900 coupled to an electronic communication device 1400. The electronic communication device 1400 can comprise one or more of, a smartphone, a smart watch, a tablet, a laptop, and the like. The display 902 can comprise a touchscreen that provides a user interface for a user to select between a spirometer function and a vaping function of the exemplary vapor device 900. In the spirometer mode, the exemplary vapor device 900 can be configured to permit a user to exhale/inhale into a mouthpiece 912 and for the exemplary vapor device 900 to perform a spirometer function. For example, the exemplary vapor device 900 can configure one or more sensors and flow tubes through the exemplary vapor device 900 to enable measurement of one or more spirometry parameters. The measurements can be received/determined and can be passed to the electronic communication device 1400 via an input/output port 910 (not shown). The electronic communication device 1400 can display results of the spirometer function. FIG. 14 illustrates the results of performing a specific spirometry analysis. The exemplary vapor device 900 can perform a forced vital capacity (FVC) measurement. This is a measurement of lung size (e.g., in liters) and represents the volume of air in the lungs that can be exhaled following a deep inhalation. The exemplary vapor device 900 can perform forced expiratory volume-one second (FEV1). This is a measure of how much air can be exhaled in one second following a deep inhalation. The exemplary vapor device 900 can then determine a FEV1/FVC ratio. This ratio represents the percent of the lung size (FVC) that can be exhaled in one second. For example, if the FEV1 is 4 and the FVC is 5, then the FEV1/FVC ratio would be 4/5 or 80%. This means the user can breathe out 80% of the inhaled air in the lungs in one second. The electronic communication device 1400 can provide a visual display of the FEV1/FVC ratio via user interface element 1404. The user interface element 1404 can display a graph from 0% to 100% and place an indicator 1406 on the graph representing the user's FEV1/FVC ratio. The user's FEV1/FVC ratio can also be provided to the user via a user interface element 1408. The user interface element 1408 can provide a textual/numerical representation of the user's FEV1/FVC ratio.

In an aspect, the exemplary vapor device 900, the electronic communication device 1400, and or a remote computing device can perform an analysis of the user's FEV1/FVC ratio and recommend one or more respiratory medications (including mixtures thereof) to improve the user's respiratory function. Once the recommended respiratory medication has been determined, if the exemplary vapor device 900 contains the respiratory medication, the exemplary vapor device 900 can calibrate the recommended dosage and prepare the calibrated dose for vaporization and subsequent inhalation by the user. The electronic communication device 1400 can communicate to the user that the dosage has been calibrated via a user interface element 1410. Once the dose has been calibrated, the user can be presented with the option to proceed with the dose via a user interface element 1412. Upon engaging an affirmative option via the user interface element 1412, the electronic communication device 1400 can pass a command to the exemplary vapor device 900 to vaporize the dose and provide the dose to the user via the mouthpiece 912. Other spirometric analyses are specifically contemplated herein.

In an aspect, disclosed herein is a system, method and device deployment of an electronic hybrid eSpirometer and respiratory medication dispenser which couples and functions symbiotically to a portable electronic communication device. The electronic spirometer is synched to the companion device and displays results from the spirometer on the device screen. The hybrid spirometer device results yield the information which may trigger a dose of medication on a prescribed basis, which may be at least one of, taken at a certain time(s) each day, taken as needed, taken in specific quantities based upon the eSpirometer results. The dosage of medication(s) needed to be taken is displayed on at least one of the system interface of the companion device or the instant device. The at least one of the system devices displays alerts to take, refill, recharge, contact a caregiver, calibrate the spirometer or medication dispenser or sign in and validate user identification. Authorized health care representatives or user proxies can send and access information to and from the user companion devices. The companion cartridge is at least one of refillable with medication, disposable, capable of working independently or as a companion device and rechargeable.

In an aspect, provided is an apparatus comprising an air intake and a vapor output, configured for receiving a user exhalation or inhalation. The apparatus can comprise a sensor, coupled to the air intake and the vapor output, configured for determining a spirometry parameter based on the received user exhalation or inhalation. The apparatus can comprise a processor, configured for determining a mixture of vaporizable respiratory medication based on the spirometry parameter. The apparatus can comprise a plurality of containers for storing vaporizable respiratory medication. The apparatus can comprise a mixing element, coupled to the processor, configured for withdrawing a selectable amount of vaporizable respiratory medication from each of the plurality of containers based on the mixture of vaporizable respiratory medication. The apparatus can comprise a mixing chamber coupled to the air intake for receiving air, the mixing element for receiving the selectable amounts of vaporizable respiratory medication. The apparatus can comprise a heating element, coupled to the mixing chamber, configured for heating the selectable amounts of vaporizable respiratory medication and the received air to generate a vapor expelled through the vapor output.

The apparatus can comprise an e-cigarette, an e-cigar, an electronic vapor modified device, a hybrid electronic communication handset coupled/integrated vapor device, a micro-sized electronic vapor device, or a robotic vapor device.

The apparatus can comprise a memory element configured for storing the mixture of vaporizable respiratory medication wherein the processor is further configured to access the stored mixture of vaporizable respiratory medication.

The apparatus can comprise a network access device configured for transmitting data representing the spirometry parameter to a remote computing device and receiving the mixture of vaporizable respiratory medication from the remote computing device.

The apparatus can comprise a cooling element coupled to the mixing chamber, configured for receiving and cooling the heated vapor and providing the cooled vapor to the vapor output. The apparatus can comprise a magnetic element coupled to the cooling element, configured for receiving and magnetizing the vapor and providing the vapor to the vapor output. The apparatus can comprise a heating casing enclosing the heating element.

The apparatus can comprise a user interface configured for displaying the spirometry parameter and for receiving a command from a user to vaporize the vaporizable respiratory medication. The apparatus can comprise an input/output port configured for communicatively coupling the apparatus with an electronic communication device. The electronic communication device can comprise one or more of a smartphone, a smart watch, a tablet, a laptop, and combinations thereof. The electronic communication device can be configured for determining the mixture of vaporizable respiratory medication based on the spirometry parameter. The electronic communication device can comprise a user interface configured for displaying the spirometry parameter and for receiving a command from a user to vaporize the vaporizable respiratory medication.

The sensor can be configured for measuring one or more spirometry parameters comprising one or more of Tidal Volume (TV), Inspiratory Reserve Volume (IRV), Expiratory Reserve Volume (ERV), Vital Capacity (VC), Forced Vital Capacity (FVC), Forced Expiratory Volume in 1 second (FEV1), Forced Inspiratory Vital Capacity (FIVC), Peak Inspiratory Flow (PIF), and/or Peak Expiratory Flow (PEF).

In another aspect, provided is an apparatus comprising a detachable vaporizer/spirometer comprising, an air intake, a vapor output, configured for receiving a user exhalation or inhalation, a sensor, coupled to the air intake and the vapor output, configured for determining a spirometry parameter based on the received user exhalation or inhalation, a processor, configured for determining a vaporizable respiratory medication based on the spirometry parameter, a container for storing the vaporizable respiratory medication, and a mixing chamber coupled to the air intake for receiving air, the container for receiving the vaporizable respiratory medication, and a heating element configured for heating the vaporizable respiratory medication and the received air to generate a heated vapor expelled through the vapor output. The apparatus can comprise an electronic communication device, coupled to the detachable vaporizer/spirometer via an input/output port, comprising, a user input interface for controlling one or more functions of the detachable vaporizer/spirometer.

The electronic communication device can comprise one or more of a smartphone, a smart watch, a tablet, a laptop, and combinations thereof. The input/output port can comprise one or more of a USB connection, a dock connector, a Portable Digital Media Interface, and combinations thereof.

The detachable vaporizer/spirometer can comprise a cooling element coupled to the mixing chamber, configured for receiving and cooling the heated vapor and providing the cooled vapor to the vapor output. The detachable vaporizer/spirometer can comprise a heating casing enclosing the heating element. The detachable vaporizer/spirometer can comprise a magnetic element coupled to the cooling element, configured for receiving and magnetizing the cooled vapor and providing the cooled vapor to the vapor output.

The user input interface for controlling one or more functions of the detachable vaporizer/spirometer can be configured to initiate one or more spirometry tests. The one or more spirometry tests can generate one or more spirometry parameters. The sensor can be configured for measuring one or more spirometry parameters comprising one or more of Tidal Volume (TV), Inspiratory Reserve Volume (IRV), Expiratory Reserve Volume (ERV), Vital Capacity (VC), Forced Vital Capacity (FVC), Forced Expiratory Volume in 1 second (FEV1), Forced Inspiratory Vital Capacity (FIVC), Peak Inspiratory Flow (PIF), and/or Peak Expiratory Flow (PEF). The electronic communication device can be configured for determining and displaying a FEV1/FVC ratio.

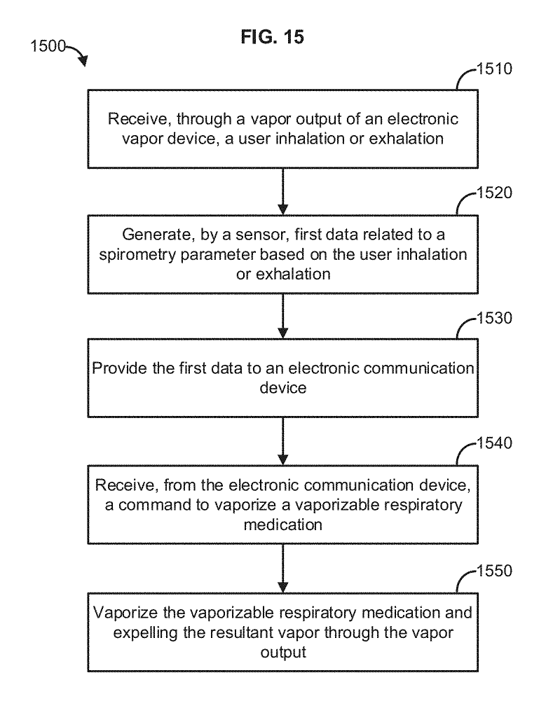

In another aspect, as illustrated in FIG. 15, provided is a method 1500 comprising receiving, through a vapor output of an electronic vapor device, a user inhalation or exhalation at 1510. The electronic vapor device can comprise one or more of a vape-bot, a micro-vapor device, a vapor pipe, e-cigarette, a hybrid handset and vapor device.

The method 1500 can comprise generating, by a sensor, first data related to a spirometry parameter based on the user inhalation or exhalation at 1520. Generating, by an electronic vapor device, first data related to a spirometry parameter based on the user inhalation or exhalation can comprise determining one or more of Tidal Volume (TV), Inspiratory Reserve Volume (IRV), Expiratory Reserve Volume (ERV), Vital Capacity (VC), Forced Vital Capacity (FVC), Forced Expiratory Volume in 1 second (FEV1), Forced Inspiratory Vital Capacity (FIVC), Peak Inspiratory Flow (PIF), and/or Peak Expiratory Flow (PEF).

The method 1500 can comprise providing the first data to an electronic communication device at 1530. The electronic communication device can comprise one or more of a smart watch, wearable technology, a smartphone, a tablet, a laptop, and a desktop. Providing the first data to an electronic communication device can comprise providing the data via a USB connection, a dock connector, a Portable Digital Media Interface, and combinations thereof.

The electronic communication device can provide the first data to a user via a display device. The method 1500 can comprise receiving, from the electronic communication device, a command to vaporize a vaporizable respiratory medication at 1540.

The method 1500 can comprise vaporizing the vaporizable respiratory medication and expelling the resultant vapor through the vapor output at 1550.

The method 1500 can further comprise transmitting the first data to a central server via one or more of cellular communication, WiFi communication, Bluetooth® communication, and satellite communication. The method 1500 can further comprise receiving, from the central server a recommended vaporizable respiratory medication; and wherein vaporizing the vaporizable respiratory medication and expelling the resultant vapor through the vapor output comprises vaporizing the recommended vaporizable respiratory medication.

In view of the exemplary systems described supra, methodologies that may be implemented in accordance with the disclosed subject matter have been described with reference to several flow diagrams. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described herein. Additionally, it should be further appreciated that the methodologies disclosed herein are capable of being stored on an article of manufacture to facilitate transporting and transferring such methodologies to computers.

Those of skill would further appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

As used in this application, the terms “component,” “module,” “system,” and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

As used herein, a “vapor” includes mixtures of a carrier gas or gaseous mixture (for example, air) with any one or more of a dissolved gas, suspended solid particles, or suspended liquid droplets, wherein a substantial fraction of the particles or droplets if present are characterized by an average diameter of not greater than three microns. As used herein, an “aerosol” has the same meaning as “vapor,” except for requiring the presence of at least one of particles or droplets. A substantial fraction means 10% or greater; however, it should be appreciated that higher fractions of small (<3 micron) particles or droplets may be desirable, up to and including 100%. It should further be appreciated that, to simulate smoke, average particle or droplet size may be less than three microns, for example, may be less than one micron with particles or droplets distributed in the range of 0.01 to 1 micron. A vaporizer may include any device or assembly that produces a vapor or aerosol from a carrier gas or gaseous mixture and at least one vaporizable material. An aerosolizer is a species of vaporizer, and as such is included in the meaning of vaporizer as used herein, except where specifically disclaimed.

Various aspects presented in terms of systems can comprise a number of components, modules, and the like. It is to be understood and appreciated that the various systems may include additional components, modules, etc. and/or may not include all of the components, modules, etc. discussed in connection with the figures. A combination of these approaches can also be used.

In addition, the various illustrative logical blocks, modules, and circuits described in connection with certain aspects disclosed, herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational aspects disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed aspects. Non-transitory computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick). Those skilled in the art will recognize many modifications may be made to this configuration without departing from the scope of the disclosed aspects.

The previous description of the disclosed aspects is provided to enable any person skilled in the art to make or use the present disclosure. Various modifications to these aspects will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. An electronic hybrid eSpirometer and respiratory medication device, wherein the device couples and functions symbiotically to a portable companion electronic communication device.

2. The device of claim 1, wherein the electronic hybrid eSpirometer is synched to the companion electronic communication device and displays results from the spirometer on a device screen.

3. The device of claim 2, wherein the results from the electronic hybrid eSpirometer yield information which may trigger a dose of medication(s) on a prescribed basis, which may be at least one of, taken at a certain time(s) each day, taken as needed, taken in specific quantities based upon the eSpirometer results.

4. The device of claim 3, wherein the dosage of indication(s) needed to be taken is displayed on at least one of a system interface of the companion electronic communication device and the electronic hybrid eSpirometer and respiratory medication device.

5. The device of claim 1, wherein the at least one of the system devices displays alerts to take, refill, recharge, contact a caregiver, calibrate the spirometer or medication dispenser or sign in and validate user identification.

6. The device of claim 1, wherein authorized health care representatives or user proxies can send and access information to and from user companion devices.

7. The device of claim 1, wherein a companion cartridge is at least one of refillable with medication, disposable, capable of working independently or as a companion device and rechargeable.

8. An apparatus comprising:
an air intake;
a vapor output, configured for receiving a user exhalation or inhalation;
a sensor, coupled to the air intake and the vapor output, configured for determining a spirometry parameter based on the received user exhalation or inhalation;
a processor, configured for determining a mixture of vaporizable respiratory medication based on the spirometry parameter;
a plurality of containers for storing vaporizable respiratory medication;
a mixing element, coupled to the processor, configured for withdrawing a selectable amount of vaporizable respiratory medication from each of the plurality of containers based on the mixture of vaporizable respiratory medication;
a mixing chamber coupled to the air intake for receiving air, the mixing element for receiving the selectable amounts of vaporizable respiratory medication; and
a heating element, coupled to the mixing chamber, configured for heating the selectable amounts of vaporizable respiratory medication and the received air to generate a vapor expelled through the vapor output.

9. The apparatus of claim 8, wherein the apparatus comprises an e-cigarette, an e-cigar, an electronic vapor modified device, a hybrid electronic communication handset coupled/integrated vapor device, a micro-sized electronic vapor device, or a robotic vapor device.

10. The apparatus of claim 8, further comprising a memory element configured for storing data related to the mixture of vaporizable respiratory medication wherein the processor is further configured to access the stored data related to the mixture of vaporizable respiratory medication.

11. The apparatus of claim 8, further comprising a network access device configured for transmitting data representing the spirometry parameter to a remote computing device and receiving the data related to the mixture of vaporizable respiratory medication from the remote computing device.

12. The apparatus of claim 8, further comprising a cooling element coupled to the mixing chamber, configured for receiving and cooling the heated vapor and providing the cooled vapor to the vapor output.

13. The apparatus of claim 12, further comprising a magnetic element coupled to the cooling element, configured for receiving and magnetizing the vapor and providing the vapor to the vapor output.

14. The apparatus of claim 8, further comprising a heating casing enclosing the heating element.

15. The apparatus of claim 8, further comprising a user interface configured for displaying the spirometry parameter and for receiving a command from a user to vaporize the vaporizable respiratory medication.

16. The apparatus of claim 8, further comprising an input/output port configured for communicatively coupling the apparatus with an electronic communication device.

17. The apparatus of claim 16, wherein the electronic communication device comprises one or more of a smartphone, a smart watch, a tablet, a laptop, and combinations thereof.

18. The apparatus of claim 16, wherein the electronic communication device is configured for determining the mixture of vaporizable respiratory medication based on the spirometry parameter.

19. The apparatus of claim 16, wherein the electronic communication device comprises a user interface configured for displaying the spirometry parameter and for receiving a command from a user to vaporize the vaporizable respiratory medication.

20. The apparatus of claim 16, wherein the sensor is configured for measuring one or more spirometry parameters comprising one or more of Tidal Volume (TV), Inspiratory Reserve Volume (IRV), Expiratory Reserve Volume (ERV), Vital Capacity (VC), Forced Vital Capacity (FVC), Forced Expiratory Volume in 1 second (FEV1), Forced Inspiratory Vital Capacity (FIVC), Peak Inspiratory Flow (PIF), and/or Peak Expiratory Flow (PEF).

21. An apparatus comprising:
a detachable vaporizer/spirometer comprising,
an air intake,
a vapor output, configured for receiving a user exhalation or inhalation,
a sensor, coupled to the air intake and the vapor output, configured for determining a spirometry parameter based on the received user exhalation or inhalation;
a processor, configured for determining a vaporizable respiratory medication based on the spirometry parameter;
a container for storing the vaporizable respiratory medication, and
a mixing chamber coupled to the air intake for receiving air, the container for receiving the vaporizable respiratory medication, and a heating element configured for heating the vaporizable respiratory medication and the received air to generate a heated vapor expelled through the vapor output; and
an electronic communication device, coupled to the detachable vaporizer/spirometer via an input/output port, comprising,
a user input interface for controlling one or more functions of the detachable vaporizer/spirometer.

22. The apparatus of claim 21, wherein the electronic communication device comprises one or more of a smartphone, a smart watch, a tablet, a laptop, and combinations thereof.

23. The apparatus of claim 21, wherein the input/output port comprises one or more of a USB connection, a dock connector, a Portable Digital Media Interface, and combinations thereof.

24. The apparatus of claim 21, wherein the detachable vaporizer/spirometer comprises a cooling element coupled to the mixing chamber, configured for receiving and cooling the heated vapor and providing the cooled vapor to the vapor output.

25. The apparatus of claim 21, wherein the detachable vaporizer/spirometer comprises a heating casing enclosing the heating element.

26. The apparatus of claim 24, wherein the detachable vaporizer/spirometer comprises a magnetic element coupled to the cooling element, configured for receiving and magnetizing the cooled vapor and providing the cooled vapor to the vapor output.

27. The apparatus of claim 21, wherein the user input interface for controlling one or more functions of the detachable vaporizer/spirometer is configured to initiate one or more spirometry tests.

28. The apparatus of claim 21, wherein the sensor is configured for measuring one or more spirometry parameters comprising one or more of Tidal Volume (TV), Inspiratory Reserve Volume (IRV), Expiratory Reserve Volume (ERV), Vital Capacity (VC), Forced Vital Capacity (FVC), Forced Expiratory Volume in 1 second (FEV1), Forced Inspiratory Vital Capacity (FIVC), Peak Inspiratory Flow (PIF), and/or Peak Expiratory Flow (PEF).

29. The apparatus of claim 28, wherein the electronic communication device is configured for determining and displaying a FEV1/FVC ratio.

30. A method comprising:
receiving, through a vapor output of an electronic vapor device, a user inhalation or exhalation;
generating, by a sensor, first data related to a spirometry parameter based on the user inhalation or exhalation;
providing the first data to an electronic communication device;
receiving, from the electronic communication device, a command to vaporize a vaporizable respiratory medication; and
vaporizing the vaporizable respiratory medication and expelling the resultant vapor through the vapor output.

31. The method of claim 30, wherein the electronic vapor device comprises one or more of a vape-bot, a micro-vapor device, a vapor pipe, e-cigarette, a hybrid handset and vapor device.

32. The method of claim 30, wherein generating, by the electronic vapor device, first data related to a spirometry parameter based on the user inhalation or exhalation comprises determining one or more of Tidal Volume (TV), Inspiratory Reserve Volume (IRV), Expiratory Reserve Volume (ERV), Vital Capacity (VC), Forced Vital Capacity (FVC), Forced Expiratory Volume in 1 second (FEV1), Forced Inspiratory Vital Capacity (FIVC), Peak Inspiratory Flow (PIF), and/or Peak Expiratory Flow (PEF).

33. The method of claim 30, wherein the electronic communication device comprises one or more of a smart watch, wearable technology, a smartphone, a tablet, a laptop, and a desktop.

34. The method of claim 30, wherein providing the first data to the electronic communication device comprises providing the data via a USB connection, a dock connector, a Portable Digital Media Interface, and combinations thereof.

35. The method of claim 30, further comprising transmitting the first data to a central server via one or more of cellular communication, WiFi communication, Bluetooth® communication, and satellite communication.

36. The method of claim 35, further comprising:
receiving, from the central server a recommended vaporizable respiratory medication; and wherein vaporizing the vaporizable respiratory medication and expelling the resultant vapor through the vapor output comprises vaporizing the recommended vaporizable respiratory medication.

37. The method of claim 30, wherein the electronic communication device provides the first data to a user via a display device.
